(19) United States
(12) Patent Application Publication
(10) Pub. No.: US 2024/0087114 A1
(43) Pub. Date: Mar. 14, 2024

(54) INFORMATION PROCESSING SYSTEM

(71) Applicant: TOTO LTD., Kitakyushu-shi, Fukuoka (JP)

(72) Inventors: Takeshi Takaki, Fukuoka (JP); Masamichi Tosaki, Fukuoka (JP); Satoko Kizuka, Fukuoka (JP); Yuta Sakai, Fukuoka (JP); Shota Fujino, Fukuoka (JP); Hitoaki Higuchi, Fukuoka (JP)

(21) Appl. No.: 18/274,222

(22) PCT Filed: Mar. 9, 2022
(86) PCT No.: PCT/JP2022/010339

§ 371 Date: Jul. 26, 2023

(30) Foreign Application Priority Data
Mar. 23, 2021 (JP) 2021-048113

(51) Int. Cl. G06T 7/00; G06T 7/60

(52) U.S. Cl. CPC G06T 7/0012; G06T 7/60; G06T 2207/30004; G06T 2207/30242

(57) ABSTRACT

An information processing system according to an embodiment is an information processing system that has a detection unit that has a sensor that is mounted on a toilet where a bowl part that receives excrement is formed where a plurality of elements are linearly arranged to detect dropping feces, a feces image acquisition unit that acquires a feces image that is based on information that is acquired in time series by the detection unit, and a determination unit that determines an amount of feces from the feces image, wherein the determination unit determines an amount of feces based on a length of feces in a dropping direction thereof on the feces image and a property of feces.

### FIELD

A disclosed embodiment(s) relate(s) to an information processing system.

### BACKGROUND

A technique has conventionally been known that determines a property and/or a volume of feces (excrement) by using an image of dropping stool (that will also be referred to as “feces” below) (see, for example, Patent Literature 1). Furthermore, an invention of a toilet seat device has been known that includes a plurality of cameras and is capable of capturing images in different directions so as to capture a shape of feces sterically (see, for example, Patent Literature 2).

### CITATION LIST

### Patent Literature

Patent Literature 1: Japanese Patent Application Publication No. 2018-146244
Patent Literature 2: Japanese Patent Application Publication No. 2017-137708

### SUMMARY

### Technical Problem

However, an acquired image that includes dropping feces (that will also be referred to as a “feces image” below) includes an influence of a dropping speed of dropping feces and/or a property of feces. Hence, in a conventional technique as described above, it is difficult to determine an amount of feces appropriately, due to an influence of a dropping speed of dropping feces and/or a property of feces, so that there is room for improvement in accuracy of determination of an amount of feces that uses a feces image.

A disclosed embodiment(s) aim(s) to provide an information processing system that improves accuracy of determination of an amount of feces that uses a feces image.

### Solution to Problem

An information processing system according to one aspect of embodiments having: a detection unit that has a sensor that is installed on a toilet where a bowl part that receives excrement is formed where a plurality of elements are linearly arranged to detect dropping feces; a feces image acquisition unit that acquires a feces image that is based on information that is acquired in time series by the detection unit; and a determination unit that determines an amount of feces from the feces image, wherein the determination unit determines an amount of feces based on a length of feces in a dropping direction thereof on the feces image and a property of feces.

Even when a length of feces (that is also referred to as a “length of a feces image”) that is included in an image (a feces image) is identical, an actual amount of feces (that is also referred to as an “amount of excreted feces”) is different depending on a property of feces (that is also referred to as a “feces property”). Hence, in an information processing device according to an aspect of an embodiment, an amount of feces is determined by using a property of feces in addition to a length of feces in a dropping direction thereof on a feces image, so that such an amount of feces is determined by taking an influence of a feces property into consideration. Therefore, it is possible for an information processing device to improve accuracy of determination of an amount of feces that uses a feces image.

In the information processing system according to one aspect of embodiments, the determination unit determines the amount of feces based on a surface area that is calculated from a width of feces in a direction that intersects with a dropping direction thereof on the feces image and the length and the property of feces.

Even when a surface area of feces (that is also referred to as a “surface area of a feces image”) that is included in an image (a feces image) is identical, an actual amount of excreted feces is different depending on a property of feces (that is also referred to as a “feces property”). Hence, in an information processing device according to an aspect of an embodiment, an amount of feces is determined by using a property of feces in addition to a surface area that is based on a length of feces in a dropping direction thereof and a width (a horizontal width) thereof on a feces image, so that such an amount of feces is determined by taking an influence of a feces property into consideration. Therefore, it is possible for an information processing device to improve accuracy of determination of an amount of feces that uses a feces image.

In the information processing system according to one aspect of embodiments, the determination unit corrects and determines the amount of feces based on a threshold of a length thereof in a dropping direction thereof on the feces image that corresponds to each of properties of the feces.

A length of a feces image is different depending on a property of feces. Hence, in an information processing device according to an aspect of an embodiment, an amount of feces is corrected and determined based on a threshold of a length of a feces image in a dropping direction thereof that corresponds to each of properties of feces, so that such an amount of feces is determined by taking an influence of a feces property into consideration. Therefore, it is possible for an information processing device to improve accuracy of determination of an amount of feces that uses a feces image.

In the information processing system according to one aspect of embodiments, the determination unit determines the amount of feces based on the property of feces that is one of two or more types of properties that are based on a hardness thereof.

In an information processing system according to an aspect of an embodiment, an amount of feces is determined by taking a property of feces that is one of two or more kinds of properties that are based on hardness thereof into consideration, so that such an amount of feces is determined by taking an influence of a feces property into consideration. Therefore, it is possible for an information processing system to improve accuracy of determination of an amount of feces that uses a feces image.

In the information processing system according to one aspect of embodiments, the determination unit corrects the length in a case where the length is a predetermined length or greater, and determines the amount of feces depending on the length after correction.

In an information processing system according to an aspect of an embodiment, an amount of feces is determined by correcting a length thereof in a case where such a length is a predetermined length or greater, so that it is possible to determine an amount of feces by correcting a length of feces appropriately. Therefore, it is possible for an information processing system to improve accuracy of determination of an amount of feces that uses a feces image.

In the information processing system according to one aspect of embodiments, the determination unit divides and derives, in a case where a plurality of bowel movements are included in a single act of excreting and a plurality of properties of feces are provided, amounts for respective properties thereof, and determines the amount of feces by using a total value of derived amounts.

In an information processing system according to an aspect of an embodiment, in a case where a plurality of properties of feces are present, an amount thereof is derived divisionally for each property and an amount of feces is determined by using a total value of derived amounts, so that it is possible to determine an amount of feces appropriately even in a case where a mixture of feces with a plurality of properties is provided. Therefore, it is possible for an information processing system to improve accuracy of determination of an amount of feces that uses a feces image. A information processing system determines an amount of feces, in a case where a plurality of feces properties are present, by, for example, dividing respective feces properties and using a total value thereof, so that it is possible to improve accuracy of determination of an amount of feces.

In the information processing system according to one aspect of embodiments, the determination unit corrects, in a case where a total length that is a total of lengths of a plurality of feces in a dropping direction thereof in a single act of excreting is a predetermined length or greater, the total length, and determines the amount of feces depending on the total length after correction.

In an information processing system according to an aspect of an embodiment, in a case where a total length that is a total of a plurality of lengths of feces in a dropping direction thereof is a predetermined length or greater, such a total length is corrected and an amount of feces is determined, so that it is possible to determine an amount of feces appropriately even in a case where a plurality of types of feces are included. Therefore, it is possible for an information processing system to improve accuracy of determination of an amount of feces that uses a feces image.

### Advantageous Effects of Invention

According to an aspect of an embodiment, it is possible to improve accuracy of determination of an amount of feces that uses a feces image.

### BRIEF DESCRIPTION OF DRAWINGS

FIG. 1 is a perspective view that illustrates an example of a configuration in a toilet room according to an embodiment.

FIG. 2 is a diagram that illustrates a configuration example of an information processing system according to an embodiment.

FIG. 3 is a block diagram that illustrates an example of a functional configuration of a toilet seat device according to an embodiment.

FIG. 4 is a perspective view that illustrates an example of a configuration of a toilet seat device according to an embodiment.

FIG. 5 is a perspective view of an essential part that illustrates a part of a configuration of a toilet seat device according to an embodiment.

FIG. 6 is a front elevation view that illustrates a part of a configuration of a toilet seat device according to an embodiment.

FIG. 7 is a block diagram that illustrates an example of a configuration of an information processing device according to an embodiment.

FIG. 8 is a diagram that illustrates an example of an acquisition method for data.

FIG. 9 is a diagram that illustrates an example of acquisition of data that is dependent on a dropping speed of feces.

FIG. 10 is a diagram that illustrates an example of acquisition of data that is dependent on a dropping speed of feces.

FIG. 11 is a diagram that illustrates an example of a feces image of soft faces.

FIG. 12 is a diagram that illustrates an example of a feces image of hard faces.

FIG. 13 is a diagram that illustrates an example of information that is used for determining an amount of feces.

FIG. 14 is a diagram that illustrates an example of a relationship between a feces property and an amount of feces.

FIG. 15 is a diagram for explaining an example of determination of an amount of feces in a case of hard feces.

FIG. 16 is a diagram for explaining an example of determination of an amount of feces in a case of hard feces.

FIG. 17 is a diagram for explaining an example of determination of an amount of feces in a case of soft feces.

FIG. 18 is a diagram that illustrates an example of correction of a length of feces.

FIG. 19 is a diagram that illustrates an example of correction of a length of feces.

FIG. 20 is a diagram that illustrates an example of a feces image that includes feces with different properties.

FIG. 21 is a diagram for explaining an example of determination of an amount of feces in a case of a plurality of feces properties.

### DESCRIPTION OF EMBODIMENTS

Hereinafter, an embodiment(s) of an information processing system as disclosed in the present application will be explained in detail with reference to the accompanying drawing(s). Additionally, this invention is not limited by an embodiment(s) as illustrated below. Although a process for determination of an amount of feces and/or a configuration for executing such a process will be explained below, various types of configurations such as an information processing system and/or a configuration in a toilet room that is/are supposed will be first explained.

### 1. Configuration of Information Processing System

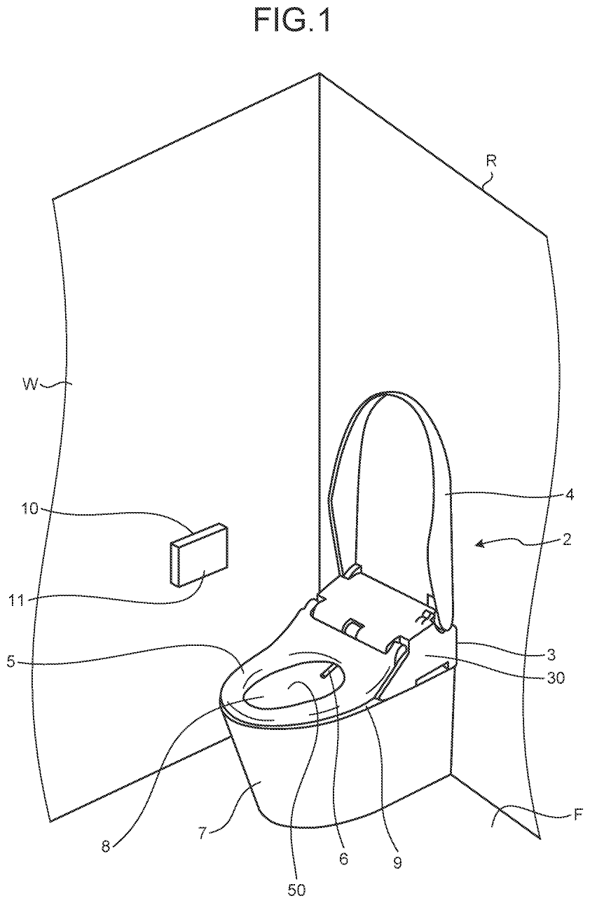

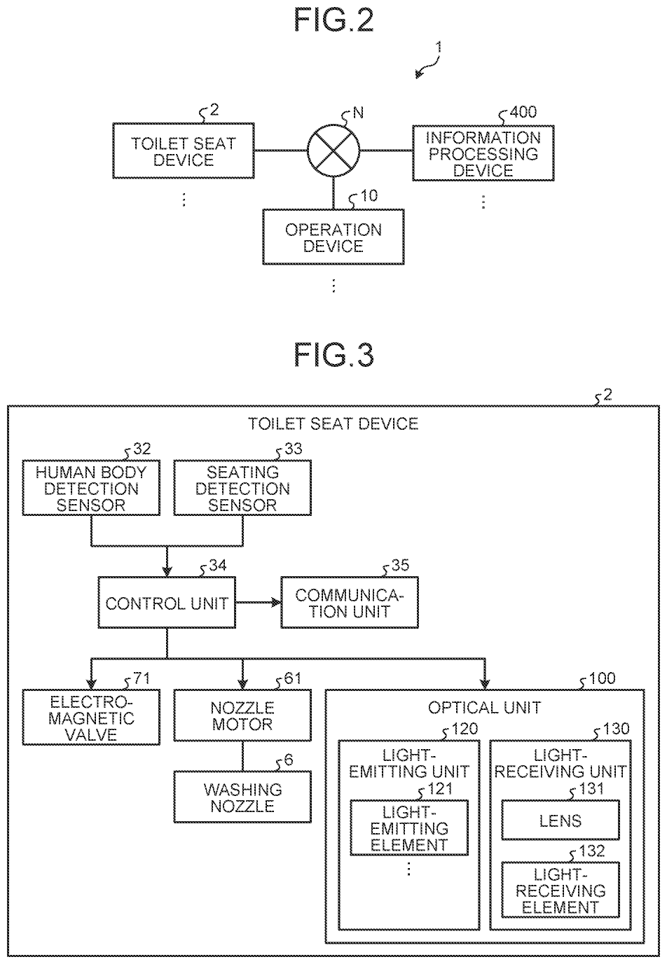

First, a configuration of an information processing system according to an embodiment will be explained with reference to FIG. 1 and FIG. 2. FIG. 1 is a perspective view that illustrates an example of a configuration in a toilet room according to an embodiment. FIG. 2 is a diagram that illustrates a configuration example of an information processing system according to an embodiment.

First, a configuration example of an information processing system 1 in a toilet room R will be explained by using FIG. 1. As illustrated in FIG. 1, in the toilet room R, a western style toilet (that will be described as a “toilet” below) 7 is installed on a floor surface F. Additionally, a direction of facing a space in the toilet room R from the floor surface F will be described as an upward one below. A toilet seat device 2 is provided on an upper part of the toilet 7.

The toilet 7 is made of, for example, a ceramic(s). A bowl part 8 is formed on the toilet 7. The bowl part 8 is a site that is provided with a downwardly dented shape and receives excrement of a user. Additionally, the toilet 7 is not limited to a floor-mounted type as illustrated in the figure and may be any type as long as the information processing system 1 is applicable thereto where is may be a type such as a wall-mounted type. The toilet 7 is provided with a rim part 9 over a whole circumference of an end part of an aperture that faces the bowl part 8. In the toilet room R, for example, a washing water tank that stores washing water may be installed near the toilet 7 or a so-called tankless type may be provided where such a washing water tank is not installed.

For example, as a washing operation unit for washing (where illustration thereof is omitted) that is provided in the toilet room R is operated by a user, toilet washing is executed by supply of washing water to the bowl part 8 of the toilet 7. A washing operation unit may be an operation lever and/or a touch operation on a toilet washing object that is displayed to an operation device 10. Additionally, a washing operation unit is not limited to toilet washing that is manually executed by a user, such as an operation lever, and may be toilet washing that is executed by human body detection of a sensor that detects a user, such as a seating sensor.

The toilet seat device 2 is attached to an upper part of the toilet 7 and includes a body part 3, a toilet lid 4, a toilet seat 5, and a washing nozzle 6. The toilet seat device 2 is mounted on an upper part of the toilet 7 where the bowl part 8 that receives excrement is formed. The toilet seat device 2 is mounted on an upper part of the toilet 7 in such a manner that the washing nozzle 6 moves into the bowl part 8 before jetting washing water. Additionally, the toilet seat device 2 may be attached so as to be attachable to and detachable from the toilet 7 or may be attached to the toilet 7 so as to be integrated therewith.

As illustrated in FIG. 1, the toilet seat 5 is formed into a ring shape that has an aperture 50 at a center thereof, and is arranged at a position that overlaps with an aperture of the toilet 7 along the rim part 9. The toilet seat 5 is seated by a user. The toilet seat 5 functions as a seating part that supports buttocks of a seating user. Furthermore, as illustrated in FIG. 1, the toilet lid 4 and the toilet seat 5 are provided in such a manner that one end part of each thereof is supported by the body part 3, and are attached thereto so as to be rotatable (openable and closable) around a supporting part of the body part 3 as a center thereof. Additionally, the toilet lid 4 is attached to the toilet seat device 2 as needed, so that the toilet seat device 2 does not have to have the toilet lid 4.

The washing nozzle 6 is a nozzle for spouting water for washing. The washing nozzle 6 is capable of jetting washing water. The washing nozzle 6 is capable of jetting washing water toward a user. The washing nozzle 6 is a nozzle for washing a private part. The washing nozzle 6 is configured to be capable of moving to or from a body cover 30 that is a housing for the body part 3 by driving of a driving source (a nozzle motor 61, etc., in FIG. 3) such as an electric motor. Furthermore, the washing nozzle 6 is connected to a water source such as a non-illustrated water pipe. Then, when the washing nozzle 6 is provided at a position (that is also referred to as a “moving position” below) where it moves to the body cover 30 that is a housing for the body part 3 as illustrated in FIG. 1, water from a water source is jetted to a body of a user so as to wash a private part thereof.

FIG. 1 illustrates a state where the washing nozzle 6 is provided at a moving position thereof. Additionally, the washing nozzle 6 may also be commonly used for washing in the toilet 7 (the bowl part 8, etc.). The washing nozzle 6 may be used so as to be switchable between a private part washing mode where a private part of a user is washed and a toilet washing mode where water is sprinkled in the toilet 7. For example, the washing nozzle 6 may be used so as to be switchable between a private part washing mode and a toilet washing mode, according to control that is executed by a control unit 34 (see FIG. 3) of the toilet seat device 2.

The operation device 10 is provided in the toilet room R. The operation device 10 is provided at a position where a user is capable of operating it. The operation device 10 is provided at a position where a user is capable of operating it at a time of seating on the toilet seat 5. In an example as illustrated in FIG. 1, the operation device 10 is arranged on a wall surface W on a right side when it is viewed by a user that seats on the toilet seat 5. Additionally, the operation device 10 may be arranged in a variety of modes that are not limited to a wall surface, as long as a user that seats on the toilet seat 5 is capable of utilizing it. For example, the operation device 10 may be provided integrally with the toilet seat device 2.

As illustrated in FIG. 2, an information processing system 1 includes a toilet seat device 2, an operation device 10, and an information processing device 400. The information processing system 1 may include a plurality of information processing devices 400, a plurality of toilet seat devices 2, and/or a plurality of operation devices 10.

The toilet seat device 2 is a device that is arranged in a toilet room R. The toilet seat device 2 transmits an acquired feces image to the information processing device 400. Additionally, a detail(s) of a configuration, etc., of the toilet seat device 2 will be described later.

The operation device 10 is connected to the toilet seat device 2 and/or the information processing device 400 through a predetermined network (a network N) so as to be communicable therewith in a wired or wireless manner. For example, the toilet seat device 2 and the operation device 10 may be connected in any way as long as transmission and receipt of information are possible, and may be connected so as to be communicable in a wired manner or may be connected so as to be communicable in a wireless manner.

The operation device 10 receives various types of operations from a user, for example, through a display surface (for example, a display screen 11) by a touch panel function. Furthermore, the operation device 10 may include a switch and/or a button and receive various types of operations through a switch, a button, etc. The display screen 11 is a display screen of a tablet terminal, etc., that is/are realized by, for example, a liquid crystal display, an organic EL (Electro-Luminescence) display, etc., and is a display device for displaying various types of information. That is, the operation device 10 receives an input of a user through the display screen 11 and also executes output to a user. The display screen 11 is a display device that displays various types of information.

The operation device 10 receives an operation of a user for stopping control that is being executed by the toilet seat device 2. The operation device 10 receives an operation of a user for starting execution of washing of a private part by the toilet seat device 2. The operation device 10 receives an instruction of a user for a washing nozzle 6. The operation device 10 receives an operation of a user for causing the toilet seat device 2 to output a predetermined sound. The operation device 10 receives an operation of a user for executing a sterilization process that sterilizes the washing nozzle 6 (see FIG. 1) of the toilet seat device 2 with sterile water. The operation device 10 receives an operation of a user for adjusting a momentum of water that is spouted by the toilet seat device 2 at a time of washing of a private part thereof. The operation device 10 receives an operation of a user for adjusting a volume of a sound that is output by the toilet seat device 2. The operation device 10 receives an operation of a user for selecting language at a time when information concerning utilization of a toilet is displayed on the operation device 10 or a sound thereof is output.

For example, the operation device 10 may display an object that receives an operation of a user as described above, on the display screen 11, and execute various types of processes, depending on contact of a user with a displayed object. For example, the operation device 10 may have a switch and/or a button that receive(s) an operation of a user as described above, and execute various types of processes, depending on contact of a user with such a switch, a button, etc. Additionally, the above is an example and the operation device 10 may receive an operation of a user that executes various types of processes.

The information processing device 400 is a computer that determines an amount of feces based on a length of feces in a dropping direction thereof on a feces image and a property of feces that is determined from such a feces image. The information processing device 400 determines an amount of feces by using a feces image that is acquired by the toilet seat device 2. The information processing device 400 is connected to the toilet seat device 2 and/or the operation device 10 through a predetermined network (a network N) such as the Internet so as to be communicable therewith in a wired or wireless manner. Additionally, the information processing device 400 may be connected to the toilet seat device 2 and/or the operation device 10 in any way as long as transmission and receipt of information are possible, and may be connected so as to be communicable in a wired manner or may be connected so as to be communicable in a wireless manner.

Additionally, the above is merely an example and it is possible to employ any form for a device configuration and arrangement of the information processing device 400 as long as it is possible to communicate with the toilet seat device 2 and/or the operation device 10 and realize a process. For example, the information processing device 400 may be a mobile terminal (device) such as a notebook personal computer that is portable by an administrator, etc., of the information processing system 1. Furthermore, the information processing device 400 may be arranged in the toilet room R.

Furthermore, the information processing device 400 may be integrated with the toilet seat device 2. In such a case, the toilet seat device 2 functions as an information processing device that executes a determination process. For example, the control unit 34 (FIG. 3) of the toilet seat device 2 may execute a determination process. Furthermore, the information processing device 400 may be integrated with a repeater (a gateway) of a predetermined network (for example, a network N). In such a case, a repeater functions as an information processing device that executes a determination process. That is, an information processing device that executes a determination process may be any device that is included in the information processing system 1. Additionally, a system configuration as described above is merely an example and the information processing system 1 may be any system configuration as long as it is possible to determine an amount of feces.

The information processing system 1 detects, for excrement (stool) of a user, which is a property of feces thereof among two or more kinds of properties that are based on hardness thereof, by various types of configurations and/or processes as described later. Although a case where whether feces is soft feces or hard feces is detected (determined) as an example of two or more kinds of properties that are based on hardness thereof will be illustrated in an example(s) as provided below, properties that are based on hardness thereof are not limited to two kinds thereof and may be three or more kinds thereof. For example, two or more kinds of properties that are based on hardness thereof may be three kinds of properties such as soft feces, normal feces, and hard feces. Furthermore, hardness of feces reflects an amount of water that is included in feces, so that two or more kinds of properties that are based on hardness thereof may be classified into “feces that is readily torn” by its own weight, with a greater amount of water and “feces that is not readily torn” by its own weight, with a less amount of water. Additionally, the information processing system 1 may detect a shape, a size, a quality, a color, etc., of excrement (stool) of a user as a feces property that is not limited to only a hardness of feces. The information processing system 1 detects excrement of a user in an optical manner. That is, the information processing system 1 is an information processing system that is capable of detecting information of excrement (stool) by an optical means. In the information processing system 1, provision of information to a terminal device such as a smartphone of a user may be executed based on a measured result.

### 2. Functional Configuration of Seating Device

Next, a functional configuration of a toilet seat device 2 will be explained with reference to FIG. 3. FIG. 3 is a block diagram that illustrates an example of a functional configuration of a seating device according to an embodiment. As illustrated in FIG. 3, the toilet seat device 2 includes a human body detection sensor 32, a seating detection sensor 33, a control unit 34, a communication unit 35, an electromagnetic valve 71, a nozzle motor 61, a washing nozzle 6, and an optical unit 100. Additionally, illustration of a part (a body part 3, a toilet seat 5, a toilet 7, etc.) of a configuration of the toilet seat device 2 as explained in FIG. 1 is omitted in FIG. 3.

For example, the human body detection sensor 32, the seating detection sensor 33, and/or the control unit 34 is/are provided on a body part 3 of the toilet seat device 2. Furthermore, the body part 3 may have a storage unit outside the control unit 34. In such a case, the toilet seat device 2 may transmit data from the control unit 34 to a storage unit and store such data in such a storage unit.

The human body detection sensor 32 has a function to detect a human body. For example, the human body detection sensor 32 is realized by a pyroelectric sensor, etc., that use(s) an infrared signal. For example, the human body detection sensor 32 may be realized by a p (micro)wave sensor, etc. Additionally, the above is an example and the human body detection sensor 32 is not limited to the above and may detect a human body by a variety of means. For example, the human body detection sensor 32 detects a person (a user, etc.) that enters a toilet room R (see FIG. 1). The human body detection sensor 32 outputs a detection signal to the control unit 34.

The seating detection sensor 33 has a function to detect seating of a person on the toilet seat device 2. The seating detection sensor 33 detects that a user is seated on a toilet seat 5. The seating detection sensor 33 is capable of detecting seating of a user on the toilet seat 5. The seating detection sensor 33 functions as a seat leaving detection sensor that detects leaving of a user from the toilet seat 5. The seating detection sensor 33 detects a state of seat leaving of a user from the toilet seat 5.

For example, the seating detection sensor 33 detects that a user is seated on the toilet seat 5, by a load sensor. For example, the seating detection sensor 33 may be an infrared light emitting and receiving type ranging sensor and detect a human body that is present near the toilet seat 5 immediately before a person (a user) is seated on the toilet seat 5 and/or a user that is seated on the toilet seat 5. Additionally, the above is an example and the seating detection sensor 33 is not limited to the above and may detect seating of a person on the toilet seat device 2 by a variety of means. The seating detection sensor 33 outputs a seating detection signal to the control unit 34.

The communication unit 35 is realized by a communication device, a communication circuit, etc., and is communicated with an information processing device 400, an operation device 10, etc. Then, the communication unit 35 is connected to a predetermined network (a network N) such as the Internet in a wired or wireless manner and executes transmission and receipt of information to/from the information processing device 400, the operation device 10, etc. The communication unit 35 is communicated with the information processing device 400, depending on control of the control unit 34. The communication unit 35 transmits a feces image that is acquired by detection that is executed by an optical unit 100 to the information processing device 400. For example, the communication unit 35 transmits a feces image that is produced by the control unit 34 to the information processing device 400. Furthermore, the communication unit 35 executes receipt of operation information that indicates an operation of a user from the operation device 10.

The control unit 34 may be, for example, a control device that controls various types of configurations and/or processes. The control unit 34 controls a nozzle motor 61, an electromagnetic valve 71, and/or the optical unit 100. The control unit 34 controls the nozzle motor 61, the electromagnetic valve 71, and/or the optical unit 100, based on a signal that is transmitted from the operation device 10. The control unit 34 controls the nozzle motor 61, based on a signal of a control instruction concerning washing of a private part that is transmitted from the operation device 10. The control unit 34 controls the nozzle motor 61 for moving the washing nozzle 6 forward and backward. The control unit 34 controls opening and closing of the electromagnetic valve 71. The control unit 34 transmits control information for controlling turning-on and/or turning-off of a light-emitting unit 120 to the optical unit 100.

The control unit 34 transmits control information for controlling a function of an electronic shutter of a light-receiving unit 130 to the optical unit 100. Additionally, an electronic shutter of the light-receiving unit 130 is a shutter type that is different from a mechanical shutter such as a so-called lens shutter and electronically controls a light-receiving element 132 (an imaging element) so as to read light exposure. That is, an electronic shutter of the light-receiving unit 130 is a so-called electronic shutter and/or an electronically controlled shatter. The control unit 34 transmits control information to the nozzle motor 61, the electromagnetic valve 71, and/or the optical unit 100 in a wired manner. Additionally, the control unit 34 may transmit control information to the nozzle motor 61, the electromagnetic valve 71, and/or the optical unit 100 in a wireless manner.

The control unit 34 causes the optical unit 100 to execute light emission and light receipt. The control unit 34 controls the optical unit 100 so as to cause the light-emitting unit 120 to emit light and cause the light-receiving unit 130 to receive light. The control unit 34 causes the optical unit 100 to execute light emission and light receipt for a period of time when seating of a user on the toilet seat 5 is detected by the seating detection sensor 33.

The control unit 34 controls light emission of the light-emitting unit 120. The control unit 34 controls electrical conduction for a light-emitting element 121 and application of a voltage to the light-receiving element 132. The control unit 34 transmits a control instruction that opens an electronic shutter to the light-receiving element 132 and executes electrical conduction for the light-emitting element 121 so as to execute light receipt control that allows light that is reflected from stool to be received. The control unit 34 controls an interval after execution of one light receipt control is started and before light receipt control next to such one light receipt control is executed, for any period of time (for example, 0.2 milliseconds or longer, etc.) within a range where a control process is possible. Additionally, the above is merely an example and a control mode of the control unit 34 may be any mode as long as it is possible for the optical unit 100 to execute desired light emission and light receipt. Furthermore, in a case where light that is emitted from the light-emitting unit 120 is provided in one wavelength band, light from the light-emitting unit 120 does not have to be blinked according to light receipt control, and may be emitted continuously. Furthermore, in a case where a color-type light-receiving element as described later is used, continuous emission may be executed even in a case where light that is emitted from the light-emitting unit 120 is provided in a plurality of wavelength bands.

Furthermore, the control unit 34 controls the toilet lid 4 and/or the toilet seat 5 as illustrated in FIG. 1. The control unit 34 controls the toilet lid 4 and/or the toilet seat 5, based on a signal that is transmitted from the operation device 10. The control unit 34 controls the toilet lid 4 based on a signal of a control instruction concerning opening and closing of a toilet lid that is transmitted from the operation device 10. The control unit 34 controls the toilet seat 5 based on a signal of a control instruction concerning opening and closing of a seating unit that is transmitted from the operation device 10. The control unit 34 transmits control information to the toilet lid 4 and/or the toilet seat 5 in a wired manner. Additionally, the control unit 34 may transmit control information to the toilet lid 4 and/or the toilet seat 5 in a wireless manner.

The control unit 34 determines whether or not entrance of a user is detected by the human body detection sensor 32. The control unit 34 determines whether or not entrance of a user to the toilet room R is detected by the human body detection sensor 32. The control unit 34 determines whether or not seating of a user is detected by the seating detection sensor 33. The control unit 34 determines whether or not seating of a user on the toilet seat 5 is detected by the seating detection sensor 33. The control unit 34 has various types of configurations such as an operation unit that executes an operation concerning control as described above and/or a storage unit. For example, the control unit 34 is realized by a variety of means such as a processor such as a CPU (Central Processing Unit), an MPU (Micro Processing Unit), and/or an ASIC (Application Specific Integrated Circuit), and/or an integrated circuit such as an FPGA (Field Programmable Gate Array).

Herein, an example of a configuration of the control unit 34 will be explained. The control unit 34 has an AD Converter, an operation processing device, an ROM (Read Only Memory), and/or a first memory.

An AD Converter is a so-called A/D Converter (an analog-digital conversion circuit) and has a function of A/D conversion that converts an analog signal to a digital signal. An AD Converter may be an analog-digital conversion circuit. For example, an AD Converter converts analog data that are received (detected) by the light-receiving unit 130 to digital data. An AD Converter may convert, among analog data, analog data where data in a predetermined range are deleted to digital data. For example, an AD Converter may leave only data that correspond to a pixel in a preset range (for example, a central predetermined range) and delete data that correspond to a pixel in a residual range. Additionally, in a case where a dedicated sensor such as a linear sensor where a number of a pixel(s) is set for excrement detection is used for the light-receiving element 132, an AD Converter coverts a whole of analog data to digital data without executing deletion of data in a predetermined range.

An operation processing device is realized by a variety of means such as a CPU and/or a microcomputer and executes various types of processes. For example, an operation processing device executes various types of processes that uses digital data that are converted by an AD Converter. An operation processing device executes various types of processes by a program (for example, various types of programs that are associated with a detection process such as a feces detection program and/or a feces property determination program) that is stored in an ROM. For example, an operation processing device is realized by executing a program that is stored in a ROM while a storage area, etc., that is/are temporarily used in such an operation processing device is/are provided as a work area(s).

An operation processing device analyzes data. An operation processing device analyzes data that are temporarily stored in a first memory. An operation processing device executes transfer of data that are received by the light-receiving unit 130 to a first memory, and analysis and deletion of such data that are stored in a first memory.

A ROM stores, for example, various types of programs that are associated with feces detection process such as a feces detection program.

A first memory is an internal memory (a storage device) that temporarily stores various types of data. A first memory stores data that are received by the light-receiving unit 130. A first memory stores digital data that are converted by an AD Converter. For example, a first memory is an SRAM (Static Random Access Memory). Additionally, a first memory is not limited to an SRAM and another RAM (Random Access Memory), such as a DRAM (Dynamic Random Access Memory), and/or a ROM that is capable of high-speed processing, such as a PROM (Programmable Read Only Memory), is/are used.

A first memory stores data according to control by an operation processing device. For example, a storage device with a storage capacity of 96 kilobytes, 512 kilobytes, etc., is used for a first memory. Data that are temporarily stored in a first memory and are received by light-receiving unit 130 include raw data (analog data) that are detected by the light-receiving unit 130 and/or data (digital data) that are processed by being A/D-converted.

Additionally, a configuration of the control unit 34 as described above is merely an example and the control unit 34 may be any configuration as long as such a configuration is capable of a desired process. Furthermore, the toilet seat device 2 has a second memory. The toilet seat device 2 stores data that are acquired by the control unit 34 in a second memory.

For example, a second memory is an external memory (a storage device) that stores various types of data. A second memory stores digital data that are acquired from the control unit 34. For example, an EEPROM (Electrically Erasable Programmable Read-Only Memory), etc., is/are used for a second memory. A second memory may be a variety of storage devices (memories) such as an SD (Secure Digital) card memory, a USB (Universal Serial Bus) memory, etc.

A second memory is capable of transferring data that are stored in a first memory. A storage area of a second memory is greater than that of a first memory. For example, a storage device with a storage capacity that is greater than that of a first memory, such as 4 gigabytes, is used for a second memory. Data that are stored in a second memory may be transmitted to an external device. The information processing system 1 may transmit data that are stored in a second memory to an external device such as a terminal device, etc., that is/are utilized by a user, in a wireless manner, by a communication device, etc., of the toilet seat device 2.

Additionally, a second memory may be provided in any of an inside of the toilet seat device 2, an outside of the toilet seat device 2, etc. For example, a second memory may be a Micro SD inside the toilet seat device 2 or may be an external memory that is provided outside the toilet seat device 2 and communicates with the toilet seat device 2 by Wi-Fi (registered trademark) (Wireless Fidelity), etc. In such a case, an operation processing device transfers data that are temporarily stored in a first memory to a second memory by communication with such a second memory that is an external memory with a storage area that is greater than that of a first memory. Additionally, communication between a second memory and the toilet seat device 2 is not limited to Wi-Fi (registered trademark) and may be a variety of communication standards, for example, communication by ZigBee (registered trademark), Bluetooth (registered trademark), etc.

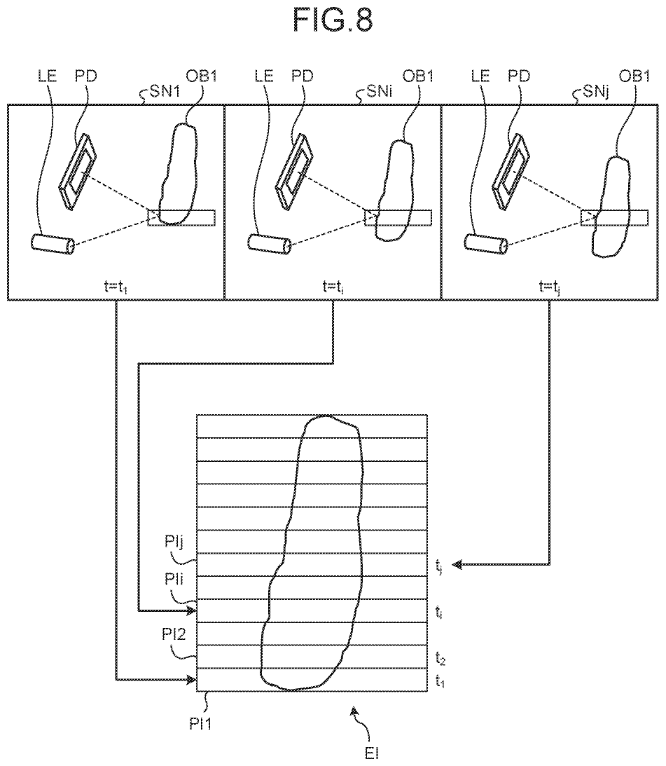

For example, the control unit 34 produces a feces image based on information that is detected by the optical unit 100. The control unit 34 arranges a plurality of one-dimensional data (linear still images) that are data that are acquired with time at a predetermined time interval(s) by the light-receiving element 132 that is a linear sensor where a plurality of elements are arranged linearly, in time sequence, so as to produce one two-dimensional image. For example, the control unit 34 produces a two-dimensional feces image based on a one-dimensional image that is detected by the optical unit 100 where such a point will be explained in FIG. 8.

The electromagnetic valve 71 has a function of a valve (a valve) that controls a flow of a fluid by an electromagnetic method. The electromagnetic valve 71 switches, for example, between supply and stop of tap water from a water supply pipe. The electromagnetic valve 71 executes control of opening and closing according to an instruction from the control unit 34.

The nozzle motor 61 is a driving source (a motor) that drives so as to move the washing nozzle 6 forward and backward. The nozzle motor 61 executes control that moves the washing nozzle 6 forward and backward relative to a body cover 30 of the body part 3. The nozzle motor 61 executes control that moves the washing nozzle 6 forward and backward according to an instruction from the control unit 34.

The optical unit 100 includes a light-emitting unit 120 and a light-receiving unit 130. The optical unit 100 functions as a detection unit (a detection device) that has a light-receiving element 132 where a plurality of elements are linearly arranged for detecting dropping feces.

The light-emitting unit 120 emits light. The light-emitting unit 120 has a light-emitting element 121 that emits light. The light-emitting unit 120 emits light toward excrement that is excreted by a user. The light-emitting unit 120 emits light toward stool that is excreted by a user. The light-emitting unit 120 emits light toward dropping feces.

The light-emitting unit 120 is provided with the light-emitting element 121 that emits light. The light-emitting unit 120 is provided with the light-emitting element 121 that emits light forward. The light-emitting unit 120 is provided with the light-emitting element 121 that emits light forward toward excrement that is excreted by a user. For example, the light-emitting element 121 is an LED (Light Emitting Diode). Additionally, the light-emitting element 121 is not limited to an LED and a variety of elements may be used.

The light-emitting unit 120 emits light forward. The light-emitting unit 120 emits light forward toward stool that is excreted by a user. The light-emitting unit 120 includes a plurality of light-emitting elements 121. The light-emitting unit 120 includes a plurality of light-emitting elements 121 that emit light. The light-emitting unit 120 emits light toward dropping stool that is excreted by a user. The light-emitting unit 120 includes a plurality of light-emitting elements 121 for emitting light with different wavelengths. Additionally, the above is merely an example, and for the light-emitting element 121 of the light-emitting unit 120, it is possible to employ any configuration for a number thereof and an emitting wavelength as long as desired emission is possible.

The light-receiving unit 130 receives light. The light-receiving unit 130 has a lens 131 and/or a light-receiving element 132 that receives light. The light-receiving unit 130 receives light that is reflected from excrement for light that is emitted from the light-emitting unit 120. The light-receiving unit 130 receives light that is reflected from stool for light that is emitted from the light-emitting unit 120. The light-receiving unit 130 receives light that is reflected from dropping stool for light that is emitted from the light-emitting unit 120.

The light-receiving unit 130 is provided with the light-receiving element 132 that receives light. The light-receiving unit 130 has the light-receiving element 132 where a plurality of elements are linearly arranged for detecting dropping feces. For example, the light-receiving element 132 is a linear sensor. For example, the light-receiving element 132 is a linear sensor where CCD (Charge Coupled Device) sensors or CMOS (Complementary Metal Oxide Semiconductor) sensors are aligned. Additionally, the light-receiving element 132 is not limited to a one-dimensional linear sensor (a one-dimensional image sensor) and various types of sensors such as a linear sensor where two or more lines are aligned and/or an area sensor (a two-dimensional image sensor) may be used.

The light-receiving unit 130 includes the lens 131 for condensing light on a front side of the light-receiving element 132. A case that is a cover for reducing or preventing entrance of light from a side other than a front side of the light-receiving element 132 is provided around the light-receiving element 132. A case that is a cover for reducing or preventing incidence of light other than light that passes through the lens 131 that is arranged on a front side thereof on the light-receiving element 132 is provided around the light-receiving element 132. A case that is a cover for reducing or preventing entrance of light from a direction of a lateral side of the light-receiving element 132 is provided around the light-receiving element 132.

A case functions as an entrance reducing or preventing cover that blocks or attenuates light from a side other than a front side of the light-receiving element 132. A case is colored with, for example, a color that does not readily reflect light, such as black, so as to reduce or prevent incidence of light that is reflected from a case, per se, on a light-receiving element. Additionally, a variety of materials, such as a resin, may be used for a case as long as it is possible to form it into a desired shape. The light-receiving unit 130 receives light that is reflected from stool for light that is emitted from the light-emitting unit 120. The light-receiving unit 130 receives light that is reflected from dropping stool for light that is emitted from the light-emitting unit 120. The light-receiving unit 130 receives light that is reflected from stool for light that is emitted from the light-emitting unit 120.

### 3. Configuration of Toilet Seat Device

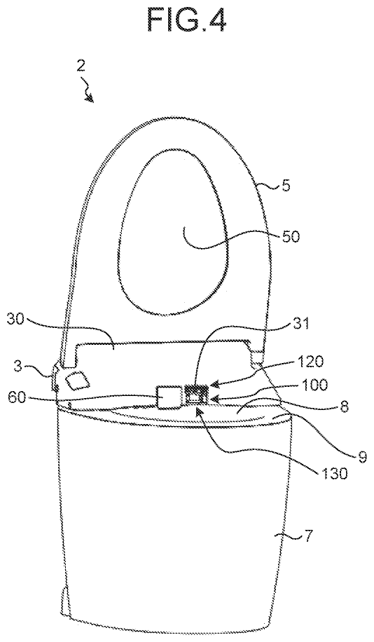

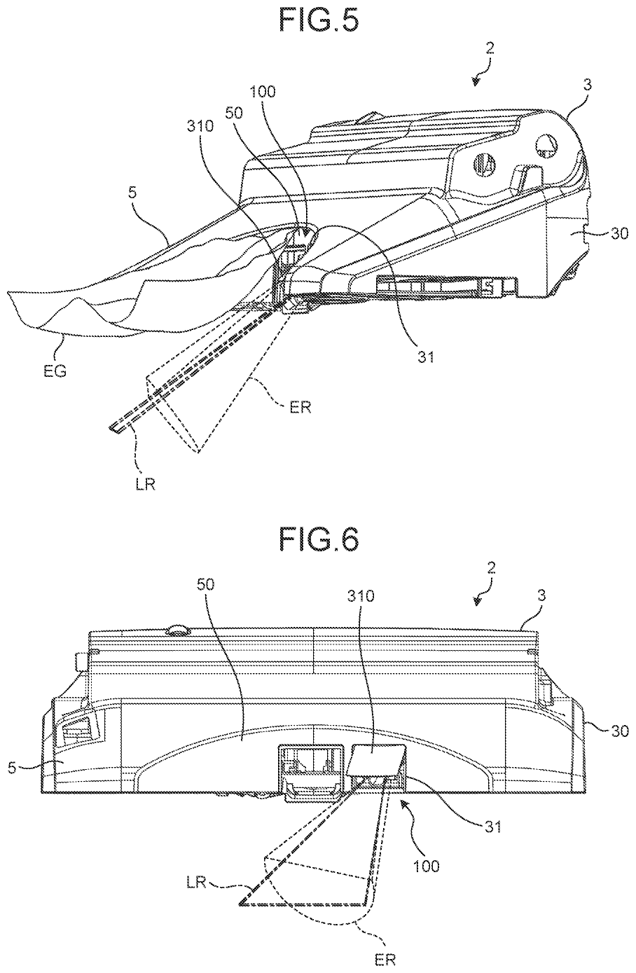

Next, a configuration of a toilet seat device 2 will be explained with reference to FIG. 4 to FIG. 6. FIG. 4 is a perspective view that illustrates an example of a seat toilet device according to an embodiment. FIG. 4 is a diagram that illustrates a state where a toilet seat 5 except for a blocking unit 310, is put up. FIG. 5 is a perspective view of an essential part that illustrates a part of a configuration of a toilet seat device according to an embodiment. FIG. 6 is a front elevation view that illustrates a part of a configuration of a toilet seat device according to an embodiment. For example, FIG. 6 is a front elevation and cross-sectional view of a toilet seat 5 in a plane that passes through an aperture 50 thereof and is orthogonal to frontward and backward directions. FIG. 5 and FIG. 6 illustrate an outline of blocking of light toward the aperture 50 of the toilet seat 5 by the blocking unit 310. Additionally, an envisaged buttock position EG in FIG. 5 virtually indicates an example of an envisaged position of buttocks of a user at a time when such a user is seated on the toilet seat 5.

As illustrated in FIG. 4, in a case where the blocking unit 310 is removed, a light-emitting unit 120 and/or a light-receiving unit 130 of an optical unit 100 is/are exposed through an aperture 31 of a body cover 30. The light-emitting unit 120 is capable of emitting light toward excrement in a toilet 7 and the light-receiving unit 130 is capable of receiving light that is reflected from excrement in the toilet 7.

Furthermore, FIG. 4 illustrates a state where a washing nozzle 6 (see FIG. 1) is provided at a position where it is housed in the body cover 30 (that will also be referred to as a “housing position” below). As illustrated in FIG. 4, in a case where the washing nozzle 6 is provided at a housing position, a lid for nozzle 60 is closed and the washing nozzle 6 is hidden behind the lid for nozzle 60. In a case where washing is executed by the washing nozzle 6, the lid for nozzle 60 is opened and the washing nozzle 6 protrudes through an aperture of the body cover 30 (an aperture that is plugged with the lid for nozzle 60 that is provided in a closed state thereof in FIG. 4), so that the washing nozzle 6 transfers to a state of forward movement thereof.

As illustrated in FIG. 5 and FIG. 6, the blocking unit 310 is arranged along an upper end part of the aperture 31 of the body cover 30 so as to block light from the light-emitting unit 120 toward the aperture 50 of the toilet seat 5. The blocking unit 310 is formed of a material with no (less) transparency. For example, the blocking unit 310 is formed of a material that is identical to that of the body cover 30.

An emission region ER indicates a region that is irradiated with light from the light-emitting unit 120 (a light-emitting element 121). Furthermore, a receiving region LR indicates a region where a light-receiving element 132 receives light. As illustrated in FIG. 6, the blocking unit 310 is positioned above the light-emitting unit 120, so that light from the light-emitting unit 120 toward the aperture 50 of the toilet seat 5, that is, light toward an upper side, is blocked. For example, the blocking unit 310 is arranged at a position above the light-emitting unit 120 in such a manner that a range of the aperture 50 of the toilet seat 5 is not included in an emission region ER. Thereby, it is possible for the toilet seat device 2 to reduce or prevent emission of light from the light-emitting unit 120 toward the aperture 50 of the toilet seat 5.

### 4. Functional Configuration of Information Processing Device

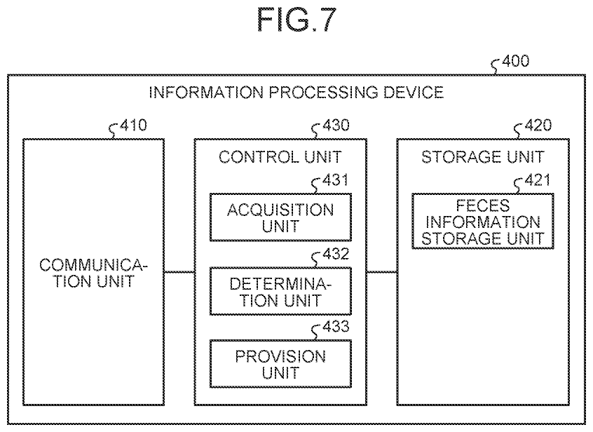

Next, a functional configuration of an information processing device will be explained with reference to FIG. 7. FIG. 7 is a block diagram that illustrates an example of a configuration of an information processing device according to an embodiment. Specifically, FIG. 7 is a block diagram that illustrates an example of a configuration of an information processing device 400 that is an example of an information processing device.

As illustrated in FIG. 7, the information processing device 400 has a communication unit 410, a storage unit 420, and a control unit 430. Additionally, the information processing device 400 may have an input unit (for example, a keyboard, a mouse, etc.) that receives various types of operations from an administrator, etc., of the information processing device 400, and/or a display unit (for example, a liquid crystal display, etc.) for displaying various types of information.

The communication unit 410 is realized by, for example, a communication circuit, etc. The communication unit 410 is connected to a network N (see FIG. 2) in a wired or wireless manner, and executes transmission and receipt of information to/from an external information processing device. For example, the communication unit 410 is connected to a network N (see FIG. 2) in a wired or wireless manner, and executes transmission and receipt of information to/from a toilet seat device 2, an operation device 10, etc.

The storage unit 420 is realized by, for example, a semiconductor memory element such as a RAM or a flash memory or a storage device such as a hard disk or an optical disk. For example, the storage unit 420 is a computer-readable recording medium that non-transiently records data, etc., that are used by an amount determination program that determines an amount of feces and/or a property determination program that determines a property of feces. As illustrated in FIG. 7, the storage unit 420 according to an embodiment has a feces information storage unit 421. Additionally, the storage unit 420 is not limited to the feces information storage unit 421 and stores various information. The feces information storage unit 421 stores various information that is used for a determination process. For example, the feces information storage unit 421 stores a threshold that is used for a determination process. Furthermore, for example, the feces information storage unit 421 stores a function that derives an amount of feces.

The feces information storage unit 421 stores information concerning detected feces (excrement). The feces information storage unit 421 stores a feces image. The feces information storage unit 421 associates and stores information concerning feces that corresponds to a feces image with such a feces image. The feces information storage unit 421 associates and stores a determination result where feces that corresponds to a feces image is determined with such a feces image. The feces information storage unit 421 stores information such as a property of feces that corresponds to a feces image and/or an amount of feces that corresponds to a feces image. Furthermore, the feces information storage unit 421 may associate and store a date and a time when a feces image is acquired, information that identifies a user that executes excreting of feces that corresponds to a feces image, etc., with such a feces image. Additionally, the above is merely an example and the feces information storage unit 421 stores various information concerning feces.

The control unit 430 is realized by, for example, a CPU, a GPU (Graphics Processing Unit), etc., where a program (for example, an amount determination program, a property determination program, etc., according to the present disclosure) that is stored in an inside of the information processing device 400 is executed while a RAM, etc., is/are provided as a work area. Furthermore, the control unit 430 is realized by, for example, an integrated circuit such as an ASIC and/or an FPGA.

As illustrated in FIG. 7, the control unit 430 has an acquisition unit 431, a determination unit 432, and a provision unit 433 and realizes or executes a function and/or an action of information processing as explained below. Additionally, an internal configuration of the control unit 430 is not limited to a configuration as illustrated in FIG. 7 and may be another configuration as long as such a configuration executes information processing as described later.

The acquisition unit 431 acquires information. The acquisition unit 431 functions as a feces image acquisition unit that acquires a feces image. The acquisition unit 431 acquires various types of information from the storage unit 420. The acquisition unit 431 receives various types of information from the toilet seat device 2 and/or the operation device 10. The acquisition unit 431 receives information concerning feces from the toilet seat device 2. The acquisition unit 431 receives an feces image (data) from the toilet seat device 2. The acquisition unit 431 is provided on the toilet seat device 2 that is mounted on an upper part of a toilet 5 where a bowl part 8 that receives excrement is formed, and acquires a feces image based on information from an optical unit 100 that has a light-receiving element 132 where a plurality of elements are linearly arranged for detecting dropping feces. The acquisition unit 431 stores an acquired feces image in the feces information storage unit 421.

The determination unit 432 executes various types of determination processes. The determination unit 432 executes a determination process by using information that is acquired from the toilet seat device 2. The determination unit 432 executes a determination process by using information that is stored in the storage unit 420. For example, the determination unit 432 determines a property of feces that corresponds to a feces image based on such a feces image. The determination unit 432 determines a property of feces that corresponds to a feces image by using such a feces image.

The determination unit 432 determines which of two or more kinds of properties that are based on a hardness a hardness of feces that corresponds to a feces image is, by using such a feces image. For example, the determination unit 432 determines whether a hardness of feces that that corresponds to a feces image is soft feces or hard feces, by using such a feces image.

The determination unit 432 determines a property of feces from a detection result from the toilet seat device 2. The determination unit 432 determines a property of feces of a user by appropriately using a variety of techniques that detect such a property of feces by an optical method. The determination unit 432 determines whether a hardness of feces is soft feces or hard feces, by appropriately using a variety of techniques concerning classification of a property of feces. For example, the determination unit 432 determines (decides) a property of feces based on various information (a feature amount) such as a length of a feces image in dropping direction thereof and/or a number of feces (a fecal pat(s)).

For example, the determination unit 432 determines that a property (a hardness) of feces that corresponds to a feces image is soft feces in a case where feces is torn (separated) so as to provide a plurality of small fecal pats in such a feces image. For example, the determination unit 432 determines that properties of a plurality of fecal pats (feces) are soft feces in a case where such a plurality of fecal pats with a length that is less than a predetermined value are provided in a feces image. For example, the determination unit 432 determines that, in a case where a fecal pat with a length that is less than a predetermined value is continuous in a feces image, a property of such a continuous fecal pat (feces) is soft feces. For example, the determination unit 432 determines that, in a case where a length of one feces (fecal pat) in a dropping direction thereof is a predetermined threshold or greater in a feces image, a property of such one feces (fecal pat) is hard feces. For example, the determination unit 432 determines that, in a case where a fecal pat with a length that is a predetermined value or greater is provided in a feces image, a property of such a fecal pat (feces) is soft feces.

Additionally, the above is merely an example and the determination unit 432 may determine a property of feces by appropriately using various information. The determination unit 432 may determine a property of feces by using a technique concerning AI (artificial intelligence). For example, the determination unit 432 may determine a property of feces by using a learning model (a property determination model) that is produced by machine learning. In such a case, a property determination model is learned by training data that preliminarily indicate classification and determination. Such training data include a plurality of combinations of a feces image and a label (ground truth information) that indicates a property (soft feces or hard feces) of fecal pat (feces) that is included in such a feces image. For example, a property determination model is a model where a feces image is input and information that indicates a property of each fecal pat (feces) that is included in an input feces image is output. For example, a property determination model is learned in such a manner that, in a case where a feces image is input, information of a label (a property of each fecal pat) that corresponds to an input feces image. Learning of a property determination model is executed by appropriately using a variety of methods concerning so-called supervised learning. In such a case, a property determination model is stored in the storage unit 420 and the determination unit 432 may determine a property of feces by using such a property determination model that is stored in the storage unit 420. For example, the information processing device 400 may execute a learning process so as to produce a property determination model.

Furthermore, the determination unit 432 does not have to execute determination of a property of feces in a case where information concerning a property of feces is acquired from another device. For example, in a case where the toilet seat device 2 executes determination of a property of feces and information concerning such a property of feces is acquired from the toilet seat device 2, the determination unit 432 does not have to execute determination of a property of feces. Additionally, in a case where information concerning a property of feces is acquired from another device, for example, a means of input to the information processing device 400 may be provided where a user acquires information concerning a determined property of feces by a mobile information terminal, etc., is input to the information processing device 400.

The determination unit 432 determines an amount of feces. The determination unit 432 determines which level an amount of feces is among a plurality of stages (levels). For example, the determination unit 432 determines which amount (stage) an amount of feces is among three stages (levels) of “less”, “normal”, and “more”. Additionally, three stages (levels) of “less”, “normal”, and “more” are merely an example and the determination unit 432 may determine which amount (stage) a stage thereof is among four or more stages (levels). For example, the determination unit 432 may determine which amount (stage) an amount of feces is among five stages (levels) of “less”, “slightly less”, “normal”, “slightly more”, and “more”. A numerical value that provides an indication of a weight and/or a volume of feces, for example, 100 g, 100 mL, etc., may be determined.

The determination unit 432 determines an amount of feces from a feces image. The determination unit 432 determines an amount of feces based on a relationship between an amount of feces and a length of feces in a dropping direction thereof in a feces image, and a property of feces that is determined from such a feces image. The determination unit 432 determines an amount of feces based on a relationship between an amount of feces and a surface area that is calculated from a width and a length of feces in a direction that intersects with a dropping direction thereof in a feces image, and a property of feces. For example, it is possible for the determination unit 432 to determine (specify) an amount of feces by using a relational expression between an amount and a length or a surface area of feces that is produced for each shape of feces, as such a length or a surface area of feces and a feces property are known. A relational expression as referred to herein is, for example, a function where a length or a surface area of feces is input and a value that indicates an amount of feces that corresponds thereto is output. For example, the determination unit 432 may select a relational expression that corresponds to a feces property among relational expressions that correspond to respective shapes of feces and determine (specify) an amount of feces by using such a relational expression and a length or a surface area of feces.

The determination unit 432 determines an amount of feces by executing length correction based on a threshold of a length in a dropping direction in a feces image that corresponds to each of properties of feces. The determination unit 432 corrects a length based on a threshold that corresponds to each of properties of feces and determines an amount of feces by a length after correction. Additionally, although length correction is herein executed based on a threshold of a length in a dropping direction in a feces image, it is not limitation and an amount of feces may be determined by correcting a surface area of feces based on a threshold of a length in a dropping direction in a feces image. Furthermore, width correction in a direction (a width direction of feces) that intersects with a dropping direction in a feces image may also be executed based on a threshold in a width direction.

The determination unit 432 determines an amount of feces based on a property of feces that is one of two or more kinds of properties that are based on a hardness thereof. The determination unit 432 corrects a length in a case where such a length is a predetermined length or greater, and determines an amount of feces by a length after correction. The determination unit 432 divides and derives amounts for respective properties in a case where a plurality of bowel movements are included in a single act of excreting (entering a toilet to exiting such a toilet) and a plurality of properties of feces are present, and determines an amount of feces by using a total value of derived amounts. The determination unit 432 corrects a total length in a case where such a total length that is a total of lengths of a plurality of feces in a dropping direction thereof in a single act of excreting is a predetermined length or greater, and determines an amount of feces by a total length after correction. Additionally, a detail(s) of determination of an amount of feces by the determination unit 432 will be described later.

The provision unit 433 provides information. The provision unit 433 transmits information to an external information processing device. For example, the provision unit 433 transmits various types of information to a mobile terminal (a user terminal) of a user that uses the toilet seat device 2, the operation device 10, and/or the toilet seat device 2. The provision unit 433 provides information that is determined by the determination unit 432 to a user terminal, etc. The provision unit 433 transmits information of an amount of feces that is determined by the determination unit 432 to a user terminal, etc.

### 5. Data Acquisition Method for Feces Image

Herein, a specific operation of an acquisition method for a feces image (data) will be explained with reference to FIG. 8. FIG. 8 is a diagram that illustrates an example of an acquisition method for data. Explanation of a point that is similar to a point as described above will be omitted appropriately.

Each element as illustrated in FIG. 8 will be explained. An object OB1 schematically illustrates stool (excrement) that is a detection (measurement) object. Furthermore, a light-receiving device PD is, for example, a light-receiving unit 130 that has a light-receiving element 132 such as a linear sensor.

Furthermore, a light-emitting device LE is a light-emitting unit 120 that has a light-emitting element 121. Additionally, although FIG. 8 explains a case where a light-emitting device LE emits one wavelength as an example for sake of simplicity of an explanation, such a light-emitting device LE may emit light with different wavelengths.

An example of FIG. 8 conceptually illustrates a process that irradiates a dropping object OB1 with light from a light-emitting device LE and acquires (produces) a feces image (a two-dimensional image) based on a result of light receipt by a light-receiving device PD. A dotted line that extends from a light-emitting device LE to an object OB1 schematically indicates irradiation of an object OB1 with light from such a light-emitting device LE and a dotted line that extends from an object OB1 to a light-receiving device PD schematically indicates light that is reflected from such an object OB1 and received by such a light-receiving device PD. Furthermore, a rectangular frame that overlaps with an object OB1 schematically indicates a (one-dimensional) range of such an object OB1 that is detected by corresponding light emission and light receipt. For example, the light-emitting unit 120 and the light-receiving unit 130 are arranged in such a manner that a position where emission and reflection of light to/from an object OB1 are executed is a position near 80 mm below an upper surface (a rim part 9) of a toilet 7 in FIG. 8. Furthermore, although a position where emission and reflection of light to/from an object OB1 are executed is herein a position near 80 mm below an upper surface (a rim part 9) of the toilet 7 in FIG. 8, it is sufficient to provide a position where emission and reflection of light to/from an object OB1 are executed and an appropriate change thereof may be executed.

In an example of FIG. 8, a scene SN1 conceptually illustrates a process that irradiates a dropping object OB1 with light from a light-emitting device LE and receives light by a light-receiving device PD, at a time t₁. Data that are acquired in a scene SN1 (a time t₁) correspond to one-dimensional image PI1 among two-dimensional images EI. That is, the toilet seat device 2 acquires (detects) a one-dimensional image PI1 by light emission and light receipt in a scene SN1 (a time t₁).

Furthermore, data that are acquired at a time t₂ correspond to a one-dimensional image PI2 among two-dimensional images EI. That is, the toilet seat device 2 acquires (detects) a one-dimensional image PI2 by light emission and light receipt at a time t₂. Data at a time t₂ are data that are acquired next to data at a time t₁. Hence, the toilet seat device 2 arranges a one-dimensional image PI2 sequentially with a one-dimensional image PI1 side by side so as to produce a two-dimensional image EI.

Furthermore, a scene SNi conceptually illustrates a process that irradiates a dropping object OB1 with light from a light-emitting device LE and receives light by a light-receiving device PD, at a time tᵢ. Data that are acquired in a scene SNi (a time tᵢ) correspond to a one-dimensional image PIi among two-dimensional images EI. That is, the toilet seat device 2 acquires (detects) a one-dimensional image PIi by light emission and light receipt in a scene SNi (a time tᵢ).

Furthermore, a scene SNj conceptually illustrates a process that irradiates a dropping object OB1 with light from a light-emitting device LE and receives light by a light-receiving device PD, at a time tⱼ. Data that are acquired in a scene SNj (a time tⱼ) correspond to a one-dimensional image PIj among two-dimensional images EI. That is, the toilet seat device 2 acquires (detects) a one-dimensional image PIj by light emission and light receipt in a scene SNj (a time tⱼ).

The toilet seat device 2 arranges one-dimensional images side by side according to an order of time when a one-dimensional image (light receiving data) is acquired, so as to produce a two-dimensional image (feces information). In FIG. 8, the toilet seat device 2 arranges one-dimensional images PI1, PI2, . . . PIi, . . . PIj, . . . side by side in an order thereof, so as to produce a two-dimensional image EI.

Additionally, although a case where light emission with one wavelength is executed has been explained as an example in an example as descried above, the toilet seat device 2 arranges, in a case where light emission with a plurality of wavelengths is executed, data (one-dimensional images) that are acquired with time for wavelengths of light emission, in a time sequence, so as to produce feces information (a two-dimensional image). On such a point, a case where light emission and light receipt of each of three light-emitting elements 121 that emit light with three different wavelengths are executed will be explained as an example.

In such a case, the toilet seat device 2 arranges light receiving data (one-dimensional images) that are obtained by executing light emission of a light-emitting element 121 (that will also be referred to as a “first light-emitting element 121”) that emits light with a first wavelength, side by side in an order of time, so as to produce a two-dimensional image that corresponds to a first light-emitting element 121. For example, the toilet seat device 2 arranges light receiving data (one-dimensional images) that are obtained by executing light emission with a first wavelength of 590 nm, etc., in a time sequence, so as to produce feces information (a first two-dimensional image) that corresponds to such a first wavelength.

Furthermore, the toilet seat device 2 arranges light receiving data (one-dimensional images) that are obtained by executing light emission of a light-emitting element 121 (that will also be referred to as a “second light-emitting element 121”) that emits light with a second wavelength, side by side in an order of time, so as to produce a two-dimensional image that corresponds to a second light-emitting element 121. For example, the toilet seat device 2 arranges light receiving data (one-dimensional images) that are obtained by executing light emission with a second wavelength of 670 nm, etc., in a time sequence, so as to produce feces information (a second two-dimensional image) that corresponds to such a second wavelength.

Furthermore, the toilet seat device 2 arranges light receiving data (one-dimensional images) that are obtained by executing light emission of a light-emitting element 121 (that will also be referred to as a “third light-emitting element 121”) that emits light with a third wavelength, side by side in an order of time, so as to produce a two-dimensional image that corresponds to a third light-emitting element 121. For example, the toilet seat device 2 arranges light receiving data (one-dimensional images) that are obtained by executing light emission with a third wavelength of 870 nm, etc., in a time sequence, so as to produce feces information (a third two-dimensional image) that corresponds to such a third wavelength.

Thus, the toilet seat device 2 produces two-dimensional images for three wavelengths that correspond to a first light-emitting element 121, a second light-emitting element 121, and a third light-emitting element 121, respectively, so that it is possible to acquire a color image. For example, the toilet seat device 2 may combine a first two-dimensional image, a second two-dimensional image, and a third two-dimensional image as described above, so as to produce a color image. Furthermore, a light-receiving element such as a linear sensor of the light-receiving unit 130 may be a color-type light-receiving element where simultaneous light emission of light-emitting elements for a plurality of colors is executed and a color of reflected light is detected by a light-receiving unit so as to produce a color image.

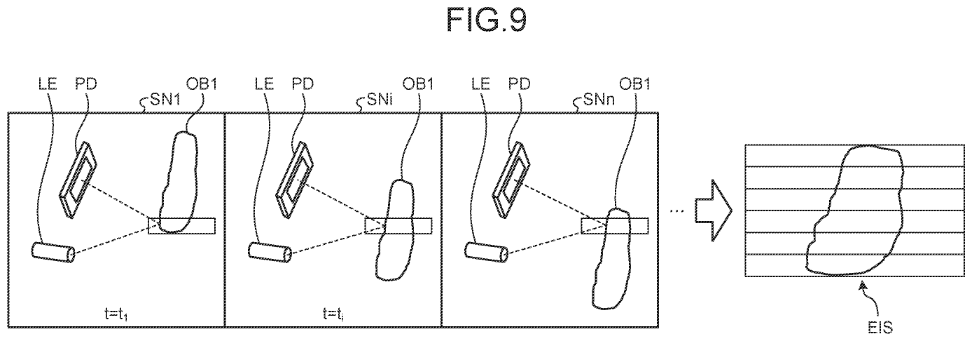

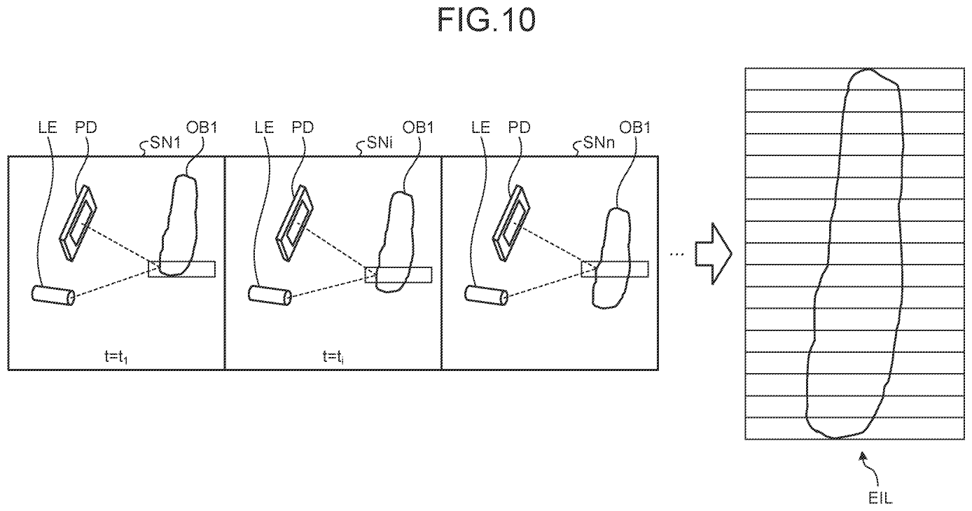

Herein, an example of acquisition of data will be explained by using FIG. 9 and FIG. 10. Additionally, an explanation of a point that is similar to that of FIG. 8 will be omitted appropriately.

First, an example of acquisition of data in a case where a dropping rate is high will be explained by using FIG. 9. FIG. 9 is diagram that illustrates an example of acquisition of data that depend on a dropping rate of feces. Specifically, FIG. 9 illustrates an example of acquisition of data in a case where a dropping rate of feces is high.

In a case where a dropping rate of feces is high, a period of time when feces passes transversely across a sensor unit is short. In an example of FIG. 9, a dropping rate of an object OB1 is high and a period of time to pass transversely across a light-receiving device PD and a light-emitting device LE is short. For example, in an example of FIG. 9, an object OB1 passes through a light-receiving device PD and a light-emitting device LE more speedily than a case of FIG. 8, so that a length of an object OB1 that passes transversely across a sensor unit (a light-receiving device PD and a light-emitting device LE) within a predetermined period of time (for example, a detection interval, etc.) in a dropping direction thereof is large. In FIG. 9, for example, a state where dropping of an object OB1 is faster than that of FIG. 8 is provided at a time tᵢ. That is, a position of an object OB1 as indicated in a scene SNi in FIG. 9 is provided below a position of an object OB1 as indicated in a scene SNi in FIG. 8.

The toilet seat device 2 arranges one-dimensional images side by side in an order of time such as a time t₁, . . . a time tᵢ, . . . , so as to produce a two-dimensional image EIS. Thus, a two-dimensional image EIS is a feces image with a length that is less than that of a two-dimensional image EI in FIG. 8.

Next, an example of acquisition of data in a case where a dropping rate is low will be explained by using FIG. 10. FIG. 10 is a diagram that illustrates an example of acquisition of data that depend on a dropping rate of feces. Specifically, FIG. 10 illustrates an example of acquisition of data in a case where a dropping rate of feces is low.

In a case where a dropping rate of feces is low, a period of time when feces passes transversely across a sensor unit is long. In an example of FIG. 10, a dropping rate of an object OB1 is low and a period of time to pass transversely across a light-receiving device PD and a light-emitting device LE is long. For example, in an example of FIG. 10, an object OB1 passes through a light-receiving device PD and a light-emitting device LE slower than a case of FIG. 8, so that a length of an object OB1 that passes transversely across a sensor unit (a light-receiving device PD and a light-emitting device LE) within a predetermined period of time (for example, a detection interval, etc.) in a dropping direction thereof is small. In FIG. 10, for example, a state where dropping of an object OB1 is later than that of FIG. 8 is provided at a time tᵢ. That is, a position of an object OB1 as indicated in a scene SNi in FIG. 10 is provided above a position of an object OB1 as indicated in a scene SNi in FIG. 8.

The toilet seat device 2 arranges one-dimensional images side by side in an order of time such as a time t₁, . . . a time tᵢ, . . . , so as to produce a two-dimensional image EIL. Thus, a two-dimensional image EIL is a feces image with a length that is greater than that of a two-dimensional image EI in FIG. 8. For example, as feces drops at an extremely low rate or feces is stopped, a one-dimensional image of an identical part thereof is continuously captured, so that data are lengthened so as to provide a feces image that is long in a dropping direction (that will also be referred to as a “length direction”) thereof.

### 6. Feces Image Example

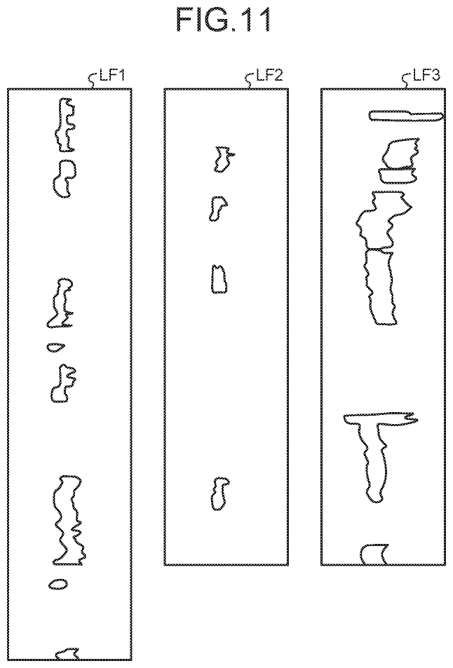

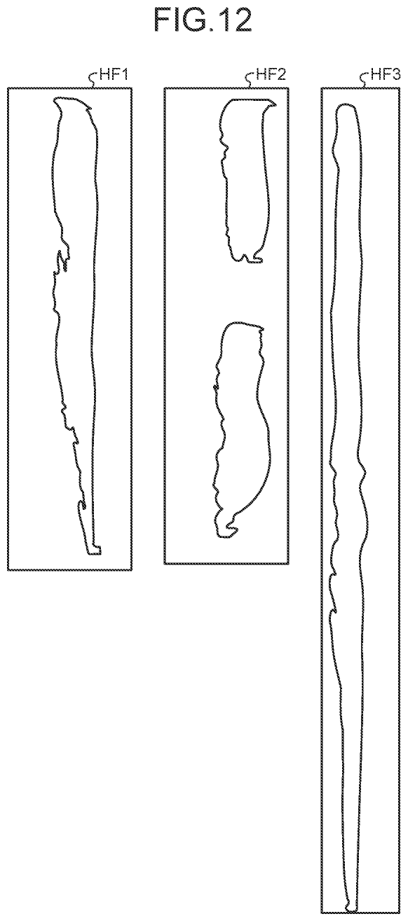

Herein, an example of a feces image will be explained by using FIG. 11 and FIG. 12. First, an example of a case of soft feces will be explained by using FIG. 11. FIG. 11 is a diagram that illustrates an example of a feces image of soft feces. Three feces images that are feces images LF1, LF2, and LF3 in FIG. 11 illustrate examples of a feces image in a case where a feces property is soft feces. As illustrated in FIG. 11, in a case where a feces property is soft feces, a water content of feces is greater, so that feces is torn by its own weight so as to (be separated into a plurality of fecal pats and) drop.

Next, an example of a case of hard feces will be explained by using FIG. 12. FIG. 12 is a diagram that illustrates an example of a feces image of hard feces. Three feces images that are feces images HF1, HF2, and HF3 in FIG. 12 illustrate examples of a feces image in a case where a feces property is hard feces. For example, a feces image HF3 illustrates a case where feces that is connected to a body and drops (moves) slowly is imaged. As illustrated in FIG. 12, in a case where a feces property is hard feces, feces is a somewhat large fecal pat. In a case where an image of dropping feces is acquired and an amount of feces is determined by such image data, an influence of a dropping rate of dropping feces and/or a property of feces is included therein, so that such an influence has to be corrected.

### 7. Determination of Amount of Feces

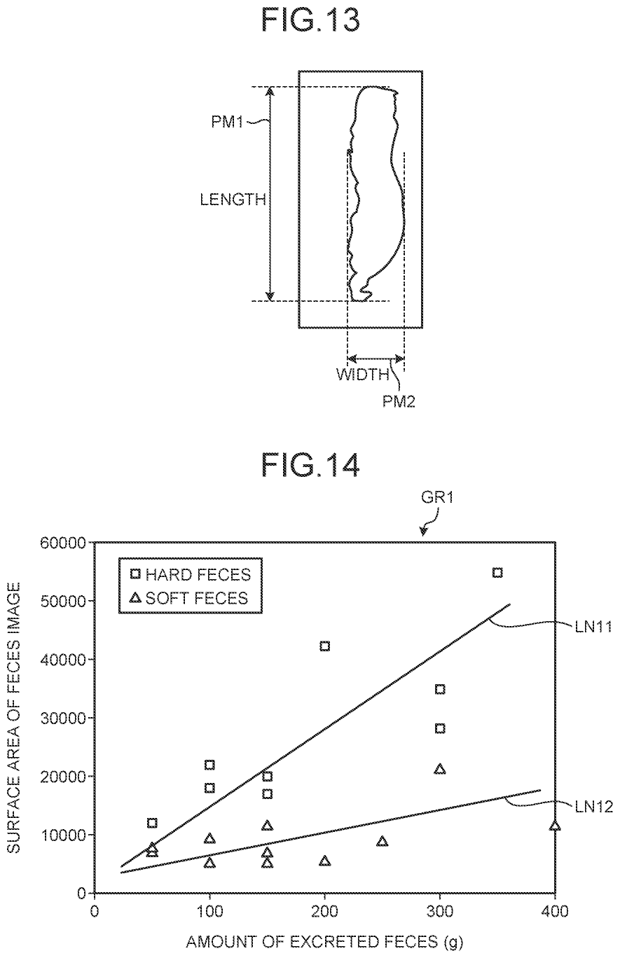

Hereinafter, determination of an amount of feces will be explained by using FIG. 13 to FIG. 21. For example, a determination process for an amount of feces as explained below is executed by a determination unit 432 of an information processing device 400. Additionally, an explanation of a point that is similar to a point as described above will be omitted appropriately.

### 7-1. Information that is Used for Determination of Amount of Feces

First, an example of information that is used for determination of an amount of feces will be explained by using FIG. 13. FIG. 13 is a diagram that illustrates an example of information that is used for determining an amount of feces. For example, the information processing device 400 uses a first parameter PM1 that indicates a length of feces in a dropping direction (a length direction) thereof as information that is used for determining an amount of feces. Furthermore, for example, the information processing device 400 uses a second parameter PM2 that indicates a width of feces in a transverse direction (a width direction) thereof as information that is used for determining an amount of feces. For example, the information processing device 400 may use an average (value) in a length direction for a second parameter PM2 that indicates a width of feces. Then, the information processing device 400 derives a surface area of feces while an average (value) of a width of feces in a length direction thereof is provided as a second parameter PM2. Furthermore, for example, a surface area may be derived by dividing feces in a dropping direction (a length direction) thereof at a predetermined interval(s) (for example, 10 pixels), calculating an average value of a width of feces at such a predetermined interval(s), and integrating a value where it is multiplied by a length of such a predetermined interval(s) (a surface area at such a predetermined interval(s)).

### 7-2. Feces Property and Amount of Feces

Next, a relationship between a feces property and an amount of feces will be explained by using FIG. 14. FIG. 14 is a diagram that illustrates an example of a relationship between a feces property and an amount of feces. Specifically, FIG. 14 illustrates a relationship between information that is obtained from a feces image, a feces property, and an amount of excreted feces (an amount of feces). An amount of excreted feces in FIG. 14 is a measurement result that is measured from a difference between body weights of a user (a subject) before and after excretion thereof. Additionally, a user (a subject) does not execute urination at a time of excretion and a degree of precision of a used weight scale is ±50 g.

A vertical axis of a graph GR1 in FIG. 14 indicates a surface area that is included in a feces image (a surface area of a feces image) and a horizontal axis indicates an amount of feces (an amount of excreted feces). A plot (data) that is represented by squares (□) in FIG. 14 indicates a measurement result in case where a feces property is hard feces. A function LN11 that is represented by a straight line in FIG. 14 is a function that indicates a relationship between a surface area of a feces image and an amount of excreted feces in a case where a feces property is hard feces. A function LN11 is a function that is derived based on a plot that is represented by squares (□) in FIG. 14. For example, a function LN11 is derived by appropriately using a method that derives a function that corresponds to (expresses) a plurality of data (for example, a regression analysis such as a least square method). For example, a function LN11 may be a function where a surface area of a feces image of feces with a property that is hard feces is input and a value that indicates an amount of feces that corresponds to such a feces image is output.

Furthermore, a plot that is represented by triangles (Δ) in FIG. 14 indicates a measurement result in case where a feces property is soft feces. A function LN12 that is represented by a straight line in FIG. 14 is a function that indicates a relationship between a surface area of a feces image and an amount of excreted feces in a case where a feces property is soft feces. A function LN12 is a function that is derived based on a plot that is represented by triangles (Δ) in FIG. 14. For example, a function LN12 is derived by appropriately using a method that derives a function that corresponds to (expresses) a plurality of data (for example, a regression analysis such as a least square method). For example, a function LN12 may be a function where a surface area of a feces image of feces with a property that is soft feces is input and a value that indicates an amount of feces that corresponds to such a feces image is output.

Additionally, the above is merely an example and a surface area of a feces image may be derived without using a length of a feces image (a first parameter PM1) and/or a width of a feces image (a second parameter PM2) that is a value that is based on a width of feces such as an average of a width of feces that is included in an image (a feces image). For example, a surface area of a feces image may be derived by calculating (counting) a number of a pixel(s). Furthermore, information that is used for derivation of an amount off feces is not limited to a surface area of a feces image and various information concerning a feces image may be used. For example, an amount of feces may be determined by using a length of a feces image and based on a relationship between such a length of a feces image and an amount of excreted feces and such a point will be described later.

A length in a dropping direction that is obtained from a feces image is different depending on a difference of properties such as hard feces and soft feces even if an amount of excreted feces is identical. Hence, setting of a threshold of a length or a surface area (an upper figure) in a dropping direction according to each property is executed, so that it is possible for the information processing device 400 to determine an amount of feces with accuracy at multiple stages.

### 7-3. Example of Determination of Amount of Hard Feces

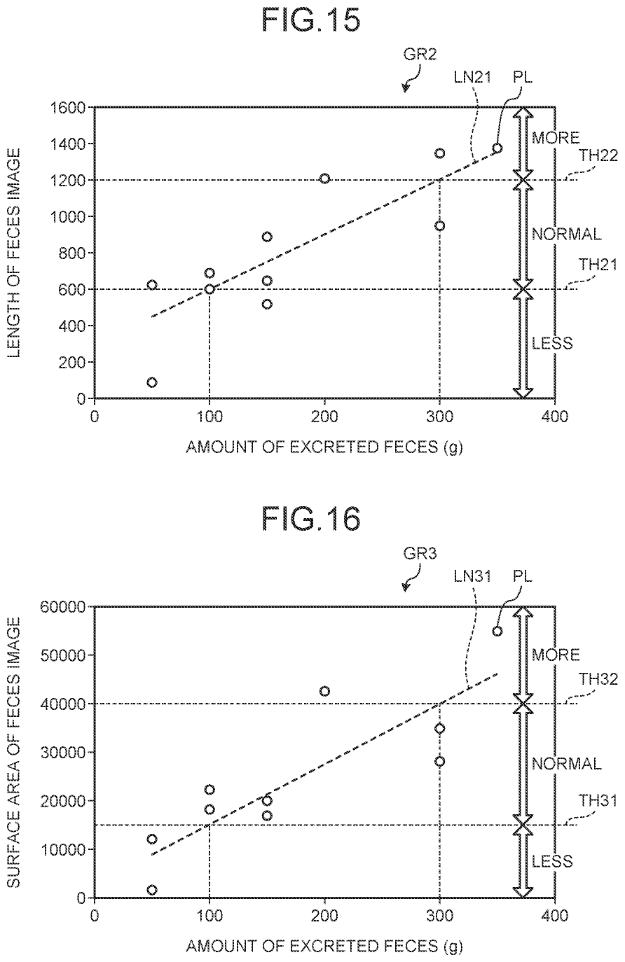

Hereinafter, FIG. 15 and FIG. 16 that are examples of determination of an amount of hard feces will be explained. First, FIG. 15 will be explained. FIG. 15 is a diagram for explaining an example of determination of an amount of feces in a case of hard feces. Specifically, FIG. 15 is a diagram that illustrates an example where an amount of feces is determined in a case of hard feces by using a length of feces. Additionally, an explanation of a point that is similar to a point as described above will be omitted appropriately.

A vertical axis of a graph GR2 in FIG. 15 indicates a length of a feces image and a horizontal axis indicates an amount of excreted feces (an amount of feces). A plot PL (data) that is represented by circles (◯) in FIG. 15 indicates a measurement result in case where a feces property is hard feces. Additionally, although only one is provided with a sign of “PL” in FIG. 15, all circles (◯) in a graph GR2 indicate a measurement result.

A function LN21 that is represented by a straight line in FIG. 15 is a function that indicates a relationship between a length of a feces image and an amount of excreted feces in a case where a feces property is hard feces. A function LN21 is a function that is derived based on a plot PL (data) that is represented by circles (◯) in FIG. 15. For example, a function LN21 is derived by appropriately using a method that derives a function that corresponds to (expresses) a plurality of data (for example, a regression analysis such as a least square method). For example, a function LN21 may be a function where a length of a feces image of feces with a property that is hard feces is input and a value that indicates an amount of feces that corresponds to such a feces image is output.

As indicated by a function LN21 in FIG. 15, a certain relationship is provided between a length of a feces image and an amount of excreted feces (an amount of feces). That is, it is possible for the information processing device 400 to estimate an amount of feces that corresponds to a feces image, from a length of such a feces image, by a function LN21.

Herein, the information processing device 400 determines an amount of feces by using a plurality of thresholds. The information processing device 400 determined an amount of feces by using two thresholds that are a first threshold and a second threshold that is a value that is greater than such a first threshold. For example, the information processing device 400 determines an amount of feces at three stages by using a first threshold and a second threshold. In such a case, for example, the information processing device 400 determines a “less” amount of feces in a case where a length of a feces image is less than a first threshold. Furthermore, the information processing device 400 determines a “normal” amount of feces in a case where a length of a feces image is a first threshold or greater and less than a second threshold. Furthermore, the information processing device 400 determines a “more” amount of feces in a case where a length of a feces image is a second threshold or greater.

An example of FIG. 15 illustrates a case of a “less” amount of feces in a case where an amount of excreted feces is less than 100 g, a “normal” amount of feces in a case where an amount of excreted feces is 100 g or greater and less than 300 g, and a “more” amount of feces in a case where an amount of excreted feces is 300 g or greater. In such a case, a length of a feces image that corresponds to an amount of feces of “100 g” is provided as a first threshold TH21 and a length of a feces image that corresponds to an amount of feces of “300 g” is provided as a second threshold TH22. A first threshold TH21 and a second threshold TH22 are determined based on information of a graph GR2. Furthermore, although an amount of feces is provided at three stages (“less”, “normal”, and “more”) herein, three stages do not have to be provided and determination may be executed with further division. Moreover, although an amount of feces is determined by using a plurality of thresholds herein, a value of a threshold and/or a number of a threshold(s) may be changed appropriately. Furthermore, a threshold does not have to be used and a length of feces may be corrected by setting a coefficient, etc., depending on a value of a length of a feces image (a length of feces).

In an example of FIG. 15, a first threshold TH21 is determined as “600”. For example, on a function LN21, a first threshold TH21 is set at a value of “600” of a length of a feces image (a length of feces) that corresponds to an amount of excreted feces of “100 g” on a horizontal axis. Furthermore, in an example of FIG. 15, a second threshold TH22 is determined as “1200”. For example, on a function LN21, a second threshold TH22 is set at a value of “1200” of a length of a feces image that corresponds to an amount of excreted feces of “300 g” on a horizontal axis. Additionally, a first threshold TH21 and a second threshold TH22 may be set preliminarily or the information processing device 400 may determine a first threshold TH21 and a second threshold TH22 by using information of a graph GR2 and/or a function LN21.

Then, the information processing device 400 determines an amount of feces while a first threshold is “600” and a second threshold is “1200”, in a case where a feces property is hard feces and information of a length of a feces image is used. For example, the information processing device 400 determines, in a case where a length of an acquired feces image is “500”, a “less” amount of feces that corresponds to such a feces image. The information processing device 400 determines, in a case where a length of an acquired feces image is “1000”, a “normal” amount of feces that corresponds to such a feces image. The information processing device 400 determines, in a case where a length of an acquired feces image is “1500”, a “more” amount of feces that corresponds to such a feces image.

Additionally, information of a feces image that is used for determination of an amount of feces is not limited to a length and may be a surface area as illustrated in FIG. 13. Such a point will be explained in FIG. 16. FIG. 16 is a diagram for explaining an example of determination of an amount of feces in a case of hard feces. Specifically, FIG. 16 is a diagram that illustrates an example where an amount of feces is determined in a case of hard feces by using a surface area of feces. Additionally, an explanation of a point that is similar to a point as described above will be omitted appropriately.

A vertical axis of a graph GR3 in FIG. 16 indicates a surface area of a feces image and a horizontal axis indicates an amount of excreted feces (an amount of feces). A plot PL (data) that is represented by circles (◯) in FIG. 16 indicates a measurement result in case where a feces property is hard feces. Additionally, although only one is provided with a sign of “PL” in FIG. 16, all circles (◯) in a graph GR3 indicate a measurement result.

A function LN31 that is represented by a straight line in FIG. 16 is a function that indicates a relationship between a surface area of a feces image and an amount of excreted feces in a case where a feces property is hard feces. A function LN31 is a function that is derived based on a plot PL (data) that is represented by circles (◯) in FIG. 16. For example, a function LN31 is derived by appropriately using a method that derives a function that corresponds to (expresses) a plurality of data (for example, a regression analysis such as a least square method). For example, a function LN31 may be a function where a surface area of a feces image of feces with a property that is hard feces is input and a value that indicates an amount of feces that corresponds to such a feces image is output.

As indicated by a function LN31 in FIG. 16, a certain relationship is provided between a surface area of a feces image and an amount of excreted feces (an amount of feces). That is, it is possible for the information processing device 400 to estimate an amount of feces that corresponds to a feces image, from a surface area of such a feces image, by a function LN31.

For example, the information processing device 400 determines an amount of feces at three stages by using a first threshold and a second threshold. In such a case, for example, the information processing device 400 determines a “less” amount of feces in a case where a surface area of a feces image is less than a first threshold. Furthermore, the information processing device 400 determines a “normal” amount of feces in a case where a surface area of a feces image is a first threshold or greater and less than a second threshold. Furthermore, the information processing device 400 determines a “more” amount of feces in a case where a surface area of a feces image is a second threshold or greater.

An example of FIG. 16 illustrates a case of a “less” amount of feces in a case where an amount of excreted feces is less than 100 g, a “normal” amount of feces in a case where an amount of excreted feces is 100 g or greater and less than 300 g, and a “more” amount of feces in a case where an amount of excreted feces is 300 g or greater. In such a case, a surface area of a feces image that corresponds to an amount of feces of “100 g” is provided as a first threshold TH31 and a surface area of a feces image that corresponds to an amount of feces of “300 g” is provided as a second threshold TH32. A first threshold TH31 and a second threshold TH32 are determined based on information of a graph GR3.

In an example of FIG. 16, a first threshold TH31 is determined as “16000”. For example, on a function LN31, a first threshold TH31 is set at a value of “16000” of a surface area of a feces image (a surface area of feces) that corresponds to an amount of excreted feces of “100 g” on a horizontal axis. Furthermore, in an example of FIG. 16, a second threshold TH32 is determined as “40000”. For example, on a function LN31, a second threshold TH32 is set at a value of “40000” of a surface area of a feces image that corresponds to an amount of excreted feces of “300 g” on a horizontal axis. Additionally, a first threshold TH31 and a second threshold TH32 may be set preliminarily or the information processing device 400 may determine a first threshold TH31 and a second threshold TH32 by using information of a graph GR3 and/or a function LN31.

For example, the information processing device 400 determines an amount of feces while a first threshold is “16000” and a second threshold is “40000”, in a case where a feces property is hard feces and information of a surface area of a feces image is used. For example, the information processing device 400 determines, in a case where a surface area of an acquired feces image is “10000”, a “less” amount of feces that corresponds to such a feces image. The information processing device 400 determines, in a case where a surface area of an acquired feces image is “30000”, a “normal” amount of feces that corresponds to such a feces image. The information processing device 400 determines, in a case where a surface area of an acquired feces image is “50000”, a “more” amount of feces that corresponds to such a feces image.

### 7-4. Example of Determination of Amount of Soft Feces

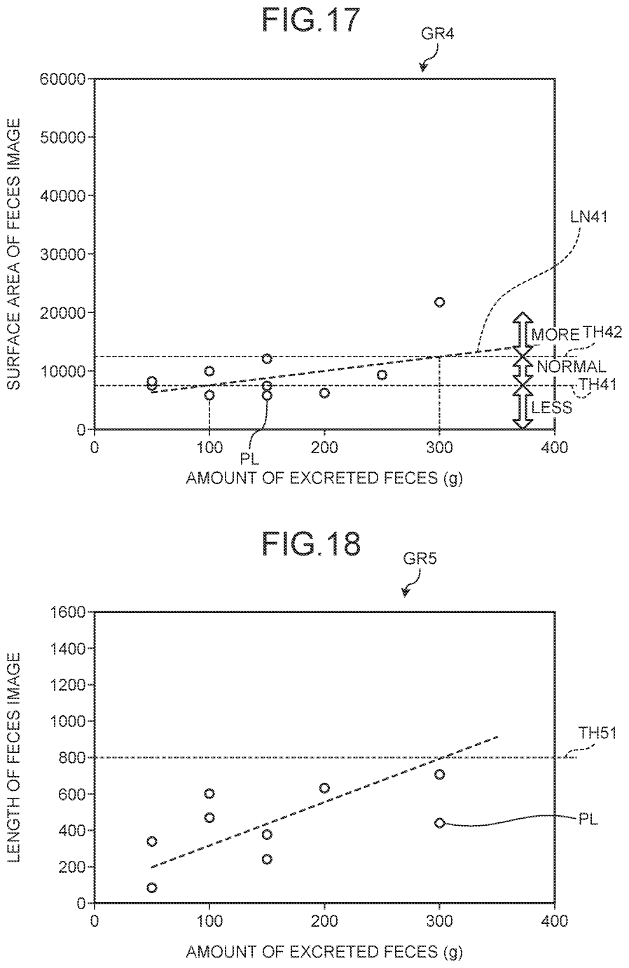

Next, FIG. 17 that is an example of determination of an amount of soft feces will be explained. FIG. 17 is a diagram for explaining an example of determination of an amount of feces in a case of soft feces. Specifically, FIG. 17 is a diagram that illustrates an example where an amount of feces is determined in a case of soft feces by using a surface area of feces. Additionally, an explanation of a point that is similar to a point as described above will be omitted appropriately.

A vertical axis of a graph GR4 in FIG. 17 indicates a surface area of a feces image and a horizontal axis indicates an amount of excreted feces (an amount of feces). A plot PL (data) that is represented by circles (◯) in FIG. 17 indicates a measurement result in case where a feces property is soft feces. Additionally, although only one is provided with a sign of “PL” in FIG. 17, all circles (◯) in a graph GR4 indicate a measurement result.

A function LN41 that is represented by a straight line in FIG. 17 is a function that indicates a relationship between a surface area of a feces image and an amount of excreted feces in a case where a feces property is soft feces. A function LN41 is a function that is derived based on a plot PL (data) that is represented by circles (◯) in FIG. 17. For example, a function LN41 is derived by appropriately using a method that derives a function that corresponds to (expresses) a plurality of data (for example, a regression analysis such as a least square method). For example, a function LN41 may be a function where a surface area of a feces image of feces with a property that is soft feces is input and a value that indicates an amount of feces that corresponds to such a feces image is output.

As indicated by a function LN41 in FIG. 17, a certain relationship is provided between a surface area of a feces image and an amount of excreted feces (an amount of feces). That is, it is possible for the information processing device 400 to estimate an amount of feces that corresponds to a feces image, from a surface area of such a feces image, by a function LN41.

For example, the information processing device 400 determines an amount of feces at three stages by using a first threshold and a second threshold. In such a case, for example, the information processing device 400 determines a “less” amount of feces in a case where a surface area of a feces image is less than a first threshold. Furthermore, the information processing device 400 determines a “normal” amount of feces in a case where a surface area of a feces image is a first threshold or greater and less than a second threshold. Furthermore, the information processing device 400 determines a “more” amount of feces in a case where a surface area of a feces image is a second threshold or greater.

An example of FIG. 17 illustrates a case of a “less” amount of feces in a case where an amount of excreted feces is less than 100 g, a “normal” amount of feces in a case where an amount of excreted feces is 100 g or greater and less than 300 g, and a “more” amount of feces in a case where an amount of excreted feces is 300 g or greater. In such a case, a surface area of a feces image that corresponds to an amount of feces of “100 g” is provided as a first threshold TH41 and a surface area of a feces image that corresponds to an amount of feces of “300 g” is provided as a second threshold TH42. A first threshold TH41 and a second threshold TH42 are determined based on information of a graph GR4.

In an example of FIG. 17, a first threshold TH41 is determined as “8000”. For example, on a function LN41, a first threshold TH41 is set at a value of “8000” of a surface area of a feces image that corresponds to an amount of excreted feces of “100 g” on a horizontal axis. Furthermore, in an example of FIG. 17, a second threshold TH42 is determined as “12000”. For example, on a function LN41, a second threshold TH42 is set at a value of “12000” of a surface area of a feces image that corresponds to an amount of excreted feces of “300 g” on a horizontal axis. Additionally, a first threshold TH41 and a second threshold TH42 may be set preliminarily or the information processing device 400 may determine a first threshold TH41 and a second threshold TH42 by using information of a graph GR4 and/or a function LN41.

For example, the information processing device 400 determines an amount of feces while a first threshold is “8000” and a second threshold is “12000”, in a case where a feces property is soft feces and information of a surface area of feces is used. For example, the information processing device 400 determines, in a case where a surface area of an acquired feces image is “5000”, a “less” amount of feces that corresponds to such a feces image. The information processing device 400 determines, in a case where a surface area of an acquired feces image is “10000”, a “normal” amount of feces that corresponds to such a feces image. The information processing device 400 determines, in a case where a surface area of an acquired feces image is “15000”, a “more” amount of feces that corresponds to such a feces image.

Additionally, information of a feces image that is used for determination of an amount of feces is not limited to a surface area and information of a length may be used as illustrated in FIG. 15 in a case of hard feces where a detailed explanation thereof will be omitted.

### 7-5. Example of Correction of Length of Feces

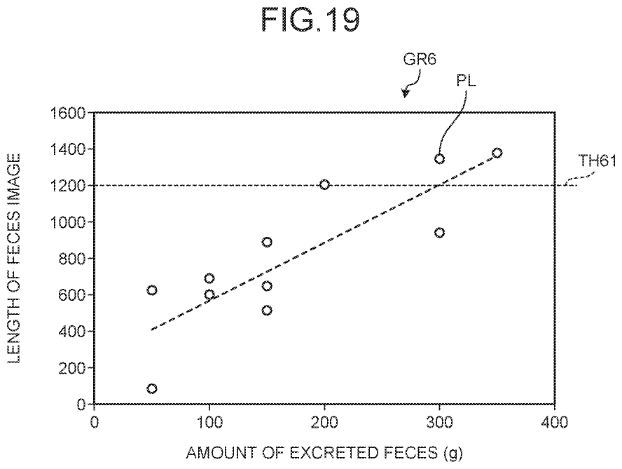

Next, FIG. 18 and FIG. 19 that are examples of correction of a length of feces will be explained. First, an example of correction of feces with a maximum length will be explained by using FIG. 18. FIG. 18 is a diagram that illustrates an example of correction of a length of feces. For example, FIG. 18 is a diagram that illustrates an example of correction of a length of feces in a case where a feces property is hard feces. Additionally, an explanation of a point that is similar to a point as described above will be omitted appropriately.

A vertical axis of a graph GR5 in FIG. 18 indicates a length of a feces image and a horizontal axis indicates an amount of excreted feces (an amount of feces). A plot PL (data) that is represented by circles (◯) in FIG. 18 indicates a measurement result in case where a feces property is hard feces. Additionally, although only one is provided with a sign of “PL” in FIG. 18, all circles (◯) in a graph GR5 indicate a measurement result.

A threshold TH51 in FIG. 18 indicates a threshold that is used for correction of a length. In an example of FIG. 18, a value of a threshold TH51 is set at “800”. In an example of FIG. 18, a length of largest feces in a dropping direction thereof is (about) “800” even at “300 g” that is determined as a “more” amount of feces (that is, a maximum level at three stages). Hence, the information processing device 400 corrects a length of largest feces while a value of “800” of a threshold TH51 is provided as an upper limit value. For example, the information processing device 400 corrects, in a case where a length of an acquired feces image is greater than a value of “800” of a threshold TH51, such a length so as to be “800”, and determines an amount of feces by using a corrected value of “800”.

Additionally, in a case where a plurality of feces (fecal pats) are included in a feces image, correction may be executed while a total of all feces (fecal pats) is a target thereof. Correction of a total length of a plurality of feces will be explained by using FIG. 19. FIG. 19 is a diagram that illustrates an example of correction of a length of feces. For example, FIG. 19 is a diagram that illustrates an example of correction of a length of feces in a case where a feces property is hard feces. Additionally, an explanation of a point that is similar to a point as described above will be omitted appropriately.

A vertical axis of a graph GR6 in FIG. 19 indicates a length of a feces image and a horizontal axis indicates an amount of excreted feces (an amount of feces). A plot PL (data) that is represented by circles (◯) in FIG. 19 indicates a measurement result in case where a feces property is hard feces. Additionally, although only one is provided with a sign of “PL” in FIG. 19, all circles (◯) in a graph GR6 indicate a measurement result.

A threshold TH61 in FIG. 19 indicates a threshold that is used for correction of a length. In an example of FIG. 19, a value of a threshold TH61 is set at “1200”. Thus, in an example of FIG. 19, in a case where a plurality of feces (fecal pats) are included in a feces image, the information processing device 400 corrects a length of largest feces while a value of “1200” of a threshold TH61 is an upper limit value. For example, the information processing device 400 corrects, in a case where a plurality of feces (fecal pats) are included in an acquired feces image and a total length of all feces (fecal pats) is greater than a value of “1200” of a threshold TH61, such a length so as to be “1200”, and determines an amount of feces by using a corrected value of “1200”.

### 7-6. Mixing of a Plurality of Feces Properties

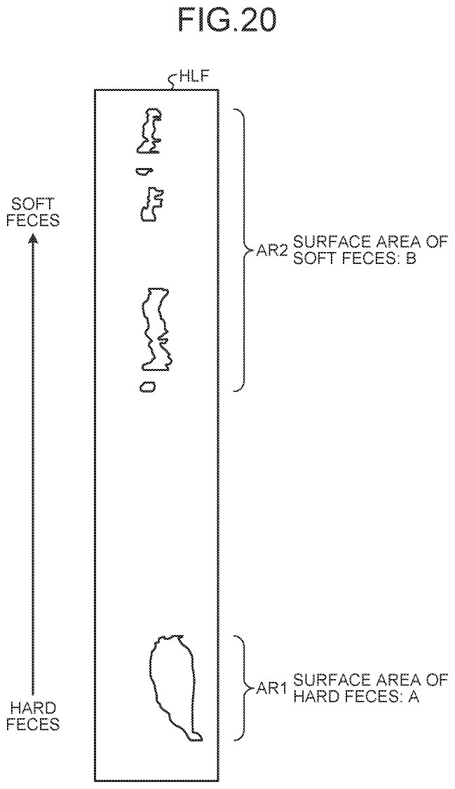

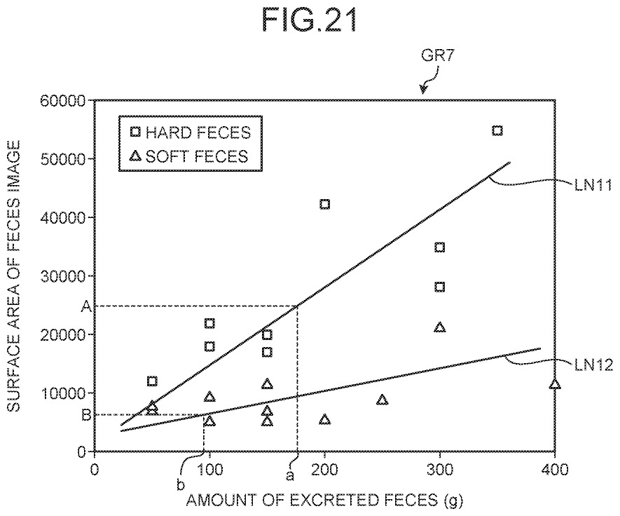

Next, a case where feces with a plurality of properties are mixed in a feces image will be explained by using FIG. 20 and FIG. 21. First, an example of a feces image where feces with a plurality of properties are mixed will be explained by using FIG. 20. FIG. 20 is a diagram that illustrates an example of a feces image that includes feces with different properties.

A feces image HLF in FIG. 20 illustrates an example of a feces image that includes faces with a feces property that is soft feces and faces with a feces property that is hard feces. A feces image HLF in FIG. 20 illustrates an example of an image where feces with a feces property that is hard feces is previously excreted and feces with a feces property that is soft feces is subsequently excreted. A feces image HLF illustrates a feces image where feces with a feces property that is hard feces is included in an area AR1 and feces with a feces property that is soft feces is included in an area AR2.

Thus, in a case where feces with a plurality of feces properties are included, the information processing device 400 derives amounts of feces for respective properties and determines an amount of feces by using a value where derived amounts of feces are totaled. Such a point will be explained by using FIG. 21. FIG. 21 is a diagram for explaining an example of determination of an amount of feces in a case of a plurality of properties. Specifically, FIG. 21 illustrates an example of determination of an amount of feces in a case where feces with a plurality of properties are mixed in a feces image while a feces image HLF in FIG. 20 is a target thereof. Additionally, an explanation of a point that is similar to a point as described above will be omitted appropriately.

A graph GR7 in FIG. 21 illustrates a graph where information for determining an amount of feces is added to a graph GR1 in FIG. 14. The information processing device 400 calculates an amount of feces with a feces property that is hard feces that is included in an area AR1 (that will also be referred to as a “first feces amount”) by using a surface area of feces (a fecal pat) with a feces property that is hard feces that is included in an area AR1 in FIG. 20 and a function LN11 in FIG. 21. A surface area (A in FIG. 20) of a feces image with a feces property that is hard feces that is included in an area AR1 is “25000”, so that the information processing device 400 calculates an amount of feces in such an area AR1 (a first feces amount) so as to be “180 g”, by a function LN11.

Furthermore, the information processing device 400 calculates an amount of feces with a feces property that is soft feces that is included in an area AR2 (that will also be referred to as a “second feces amount”) by using a surface area of feces (a fecal pat) with a feces property that is soft feces that is included in an area AR2 in FIG. 20 and a function LN12 in FIG. 21. A surface area (B in FIG. 20) of a feces image with a feces property that is soft feces that is included in an area AR2 is “8000”, so that the information processing device 400 calculates an amount of feces in such an area AR2 (a second feces amount) so as to be “95 g”, by a function LN12.

Then, the information processing device 400 totals a calculated first feces amount and second faces amount so as to derive an amount of whole feces. In an example of FIG. 21, the information processing device 400 calculates an amount of whole feces so as to be “275 (=180+95) g”. The information processing device 400 determines a derived amount of whole feces in light of a determination criterion. For example, an amount of “275 g” of whole feces in a feces image HLF in FIG. 20 is 100 g or greater and less than 300 g, so that the information processing device 400 determines a “normal” amount of feces. Thus, it is possible for the information processing device 400 to determine an amount of feces appropriately even in a case where a plurality of feces properties are mixed.

Although a case where an amount of feces is determined by using a weight of feces is explained as an example in an example as described above, the information processing device 400 may use various information that is not limited to a weight of feces as long as it is possible to determine an amount of feces. For example, the information processing device 400 may determine an amount of feces by using a volume of feces. In such a case, the information processing device 400 may determine an amount of feces by using, for example, a relational expression (a function) between a volume of feces and a length or a surface area of feces. The information processing device 400 may calculate a volume of feces from a relational expression and a length or a surface area of feces and determine (specify) an amount of feces based on a calculated volume of feces.

Additionally, it is possible to combine respective embodiments and variations as described above appropriately as long as processing contents thereof are not inconsistent.

It is possible for a person(s) skilled in the art to readily derive an additional effect(s) and/or variation(s). Hence, a broader aspect(s) of the present invention is/are not limited to a specific detail(s) and a representative embodiment(s) as illustrated and described above. Therefore, various modifications are possible without departing from the spirit or scope of a general inventive concept that is defined by the appended claim(s) and an equivalent(s) thereof.

### REFERENCE SIGNS LIST

R toilet room
1 information processing system
2 toilet seat device
3 body part
30 body cover
31 aperture
310 blocking unit
32 human body detection sensor
33 seating detection sensor
34 control unit (control device)
4 toilet lid
5 toilet seat
6 washing nozzle
60 lid for nozzle
7 western style toilet (toilet)
71 electromagnetic valve
8 bowl part
9 rim part
10 operation device
11 display screen
100 optical unit
120 light-emitting unit
121 light-emitting element
130 light-receiving unit
131 lens
132 light-receiving element
400 information processing device
410 communication unit
420 storage unit
421 feces information storage unit
430 control unit
431 acquisition unit (feces image acquisition unit)
432 determination unit
433 provision unit

What is claimed is:

1. An information processing system having:
a detection unit that has a sensor that is installed on a toilet where a bowl part that receives excrement is formed where a plurality of elements are linearly arranged to detect dropping feces;
a feces image acquisition unit that acquires a feces image that is based on information that is acquired in time series by the detection unit; and
a determination unit that determines an amount of feces from the feces image, wherein
the determination unit determines an amount of feces based on a length of feces in a dropping direction thereof on the feces image and a property of feces.

2. The information processing system according to claim 1, wherein
the determination unit determines the amount of feces based on a surface area that is calculated from a width of feces in a direction that intersects with a dropping direction thereof on the feces image and the length and the property of feces.

3. The information processing system according to claim 1, wherein
the determination unit corrects and determines the amount of feces based on a threshold of a length thereof in a dropping direction thereof on the feces image that corresponds to each of properties of the feces.

4. The information processing system according to any ono claim 1, wherein
the determination unit determines the amount of feces based on the property of feces that is one of two or more types of properties that are based on a hardness thereof.

5. The information processing system according to claim 1, wherein
the determination unit corrects the length in a case where the length is a predetermined length or greater, and determines the amount of feces depending on the length after correction.

6. The information processing system according to claim 1, wherein
the determination unit divides and derives, in a case where a plurality of bowel movements are included in a single act of excreting and a plurality of properties of feces are provided, amounts for respective properties thereof, and determines the amount of feces by using a total value of derived amounts.

7. The information processing system according to claim 1, wherein
the determination unit corrects, in a case where a total length that is a total of lengths of a plurality of feces in a dropping direction thereof in a single act of excreting is a predetermined length or greater, the total length, and determines the amount of feces depending on the total length after correction.
